(19) United States
(12) Patent Application Publication
(10) Pub. No.: US 2005/0227974 A9
(43) Pub. Date: Oct. 13, 2005

(54) Aminoalkyl-substituted aryl compounds and their use as sodium channel blockers

(75) Inventors: Qun Sun, Princeton, NJ (US); Donald J. Kyle, Newtown, PA (US)

Correspondence Address: STERNE, KESSLER, GOLDSTEIN & FOX PLLC, 1100 NEW YORK AVENUE, N.W., WASHINGTON, DC, 20005, US

(73) Assignee: Euro-Celtique S.A.

(21) Appl. No.: 10/630,763

(22) Filed: Jul. 31, 2003

Related U.S. Application Data
(60) Provisional application No. 60/399,697, filed on Aug. 1, 2002.

(51) Int. Cl. A61K 31/54; A61K 31/537; A61K 31/495; A61K 31/421; A61K 31/426; A61K 31/445; A61K 31/40

(52) U.S. Cl. 514/227.5; 514/231.2; 514/252.12; 514/317; 514/408; 514/365; 514/374; 514/649; 514/524; 544/59; 544/399; 544/162; 546/229; 548/146; 548/215; 548/577; 564/336

(57) ABSTRACT

This invention relates to a method of treating disorders responsive to the blockade of sodium ion channels using novel aminoalkyl-substituted aryl compounds of Formula I: 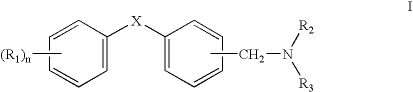
or a pharmaceutically-acceptable salt or solvate thereof, wherein n, X, R₁, R₂ and R₃ are defined in the specification. The invention is also directed to the use of compounds of Formula I for the treatment of neuronal damage following global or focal ischemia, for the treatment or prevention of neurodegenerative conditions such as amyotrophic lateral sclerosis (ALS), and for the treatment, prevention or amelioration of acute or chronic pain, neuropathic pain or surgical pain, as antitinnitus agents, as anticonvulsants, and as antimanic depressants, as local anesthetics, as antiarrhythmics and for the treatment or prevention of diabetic neuropathy.

### CROSS-REFERENCE TO RELATED APPLICATIONS

This application claims the benefit under 35 U.S.C. § 119(e) of U.S. Provisional Application No. 60/399,697, filed Aug. 1, 2002, which is hereby incorporated by reference in its entirety.

### BACKGROUND OF THE INVENTION

1. Field of the Invention

This invention is in the field of medicinal chemistry. In particular, the invention relates to novel aminoalkyl-substituted aryl compounds, and the discovery that these compounds are blockers of sodium (Na⁺) channels.

2. Related Art

Several classes of therapeutically useful drugs, including local anesthetics such as lidocaine and bupivacaine, antiarrhythmics such as propafenone and amioclarone, and anticonvulsants such as lamotrigine, phenytoin and carbamazepine, have been shown to share a common mechanism of action by blocking or modulating Na⁺ channel activity (Catterall, W. A., Trends Pharmacol. Sci. 8:57-65 (1987)). Each of these agents is believed to act by interfering with the rapid influx of Na⁺ ions.

Recently, other Na⁺ channel blockers such as BW619C89 and lifarizine have been shown to be neuroprotective in animal models of global and focal ischemia (Graham et al, J. Pharmacol. Exp. Ther. 269:854-859 (1994); Brown et al., British J. Pharmacol. 115:1425-1432 (1995)).

The neuroprotective activity of Na⁺ channel blockers is due to their effectiveness in decreasing extracellular glutamate concentration during ischemia by inhibiting the release of this excitotoxic amino acid neurotransmitter. Studies have shown that unlike glutamate receptor antagonists, Na⁺ channel blockers prevent hypoxic damage to mammalian white matter (Stys et al., J. Neurosci. 12:430-439 (1992)). Thus, they may offer advantages for treating certain types of strokes or neuronal trauma where damage to white matter tracts is prominent.

Another example of clinical use of a Na⁺ channel blocker is riluzole. This drug has been shown to prolong survival in a subset of patients with ALS (Bensimm et al., New Engl. J. Med. 330:585-591 (1994)) and has subsequently been approved by the FDA for the treatment of ALS. In addition to the above-mentioned clinical uses, carbamazepine, lidocaine and phenytoin are occasionally used to treat neuropathic pain, such as from trigeminal neurologia, diabetic neuropathy and other forms of nerve damage (Taylor and Meldrum, Trends Pharmacol. Sci. 16:309-316 (1995)), and carbamazepine and lamotrigine have been used for the treatment of manic depression (Denicott et al., J. Clin. Psychiatry 55:70-76 (1994)). Furthermore, based on a number of similarities between chronic pain and tinnitus, (Moller, A. R. Am. J. Otol. 18:577-585 (1997); Tonndorf, J. Hear. Res. 28:271-275 (1987)) it has been proposed that tinnitus should be viewed as a form of chronic pain sensation (Simpson, J. J. and Davies, E. W. Tips. 20:12-18 (1999)). Indeed, lignocaine and carbamazepine have been shown to be efficacious in treating tinnitus (Majumdar, B. et al Clin. Otolaryngol. 8:175-180 (1983); Donaldson, I. Laryngol. Otol. 95:947-951 (1981)).

It has been established that there are at least five to six sites on the voltage-sensitive Na⁺ channels which bind neurotoxins specifically (Catterall, W. A., Science 242:50-61 (1988)). Studies have further revealed that therapeutic antiarrhythmics, anticonvulsants and local anesthetics whose actions are mediated by Na⁺ channels, exert their action by interacting with the intracellular side of the Na⁺ channel and allosterically inhibiting interaction with neurotoxin receptor site 2 (Catterall, W. A., Ann. Rev. Pharmacol. Toxicol. 10:15-43 (1980)).

A need exists in the art for novel compounds that are potent blockers of sodium channels, and are therefore useful for treating a variety of central nervous system conditions, including pain.

### SUMMARY OF THE INVENTION

The present invention is related to the discovery that aminoalkyl-substituted aryl compounds represented by Formula I act as blockers of sodium (Na⁺) channels.

One aspect of the present invention is directed to treating disorders responsive to the blockade of sodium channels in a mammal suffering from excess activity of said channels, by administering an effective amount of a compound of Formula I, which acts as a blocker of sodium channels.

A further aspect of the present invention is to provide a method for treating, preventing or ameliorating neuronal loss following global and focal ischemia; treating, preventing or ameliorating pain including acute and chronic pain, and neuropathic pain; treating, preventing or ameliorating convulsions or neurodegenerative conditions; treating, preventing or ameliorating manic depression or diabetic neuropathy; using as local anesthetics and antiarrhythmics, and treating tinnitus by administering a compound of Formula I to a mammal in need of such treatment or use.

Additionally, the present invention is directed to novel aminoalkyl-substituted aryl compounds of Formula I.

Also, the present invention provides for pharmaceutical compositions useful for treating disorders responsive to the blockade of sodium ion channels, containing an effective amount of a compound of Formula I in a mixture with one or more pharmaceutically-acceptable carriers or diluents.

Additional embodiments and advantages of the, invention will be set forth in part in the description that follows, and in part will be obvious from the description, or can be learned by practice of the invention. The embodiments and advantages of the invention will be realized and attained by means of the elements and combinations particularly pointed out in the appended claims.

It is to be understood that both the foregoing general description and the following detailed description are exemplary and explanatory only, and are not restrictive of the invention as claimed.

### DETAILED DESCRIPTION OF THE INVENTION

The present invention arises out of the discovery that aminoalkyl-substituted aryl compounds of Formula I act as blockers of Na⁺ channels. Thus, in view of this discovery, a first aspect of the present invention is directed to a method of treating disorders responsive to the blockade of sodium ion channels using novel aminoalkyl-substituted aryl compounds of Formula I.

The aminoalkyl-substituted aryl compounds used in the first aspect of the present invention are represented by Formula I: 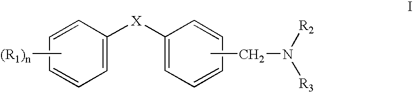
or a pharmaceutically-acceptable salt or solvate thereof, wherein:
R₁ is at each occurrence independently selected from hydrogen, halogen, optionally-substituted C₁₋₆ alkyl, amino, nitro and cyano;
n is an integer from 1 to 3;
X is —O—, —S—, —NH—, —NHCH₂—, —CH₂NH—, —CH₂—, —CH₂O—, —OCH₂—, —CH₂S— or —SCH₂—;
R₂ is 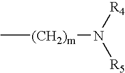
   wherein:
m is an integer from 2 to 4;
R₄ and R₅ are independently selected from hydrogen and optionally-substituted C₁₋₆ alkyl; or R₄ and R₅ together with the nitrogen to which they are attached form a ring having 4 or 5 carbon atoms, which ring optionally contains 1 or 2 additional heteroatoms independently selected from —O—, —S—, and —NR₆—, wherein R₆ is hydrogen, C₁₋₆ alkyl, C₁₋₆ haloalkyl or C₁₋₆ hydroxyalkyl; and
R₃ is hydrogen, optionally-substituted C₁₋₆ alkyl, 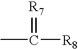
   wherein:
R₇ is oxygen or sulfur; and
R₈ is selected from optionally-substituted C₁₋₆ alkyl, an optionally-substituted C₃₋₈ carbocyclic ring system and optionally-substituted C₆₋₁₀ aryl, or R₃ is 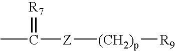
   wherein:
R₇ is oxygen or sulfur;
Z is —O— or —NH—;
p is an integer from zero to 4; and
R₉ is selected from optionally-substituted C₁₋₆ alkyl, an optionally-substituted C₃₋₈ carbocyclic ring system, optionally-substituted C₆₋₁₀ aryl, optionally-substituted heteroaryl and optionally-substituted heterocycle, wherein the heterocycle is saturated or partially unsaturated.

When the point of attachment of a ring to another moiety is not specified, e.g., where the connecting bond is drawn to the center of the ring, the point of attachment is at any available position on the ring, unless otherwise specified. For example, when n is 1, R₁ can be ortho, meta or para on the benzene ring relative to X; when n is 2, the two R₁ substituents can be positioned 2,3-, 2,4-, 2,5-, 2,6-, 3,4- or 3,5- on the benzene ring relative to X; and so forth

For the aminoalkyl-substituted aryl compounds of Formula I, the amine component is attached, through a methylene moiety, to the phenyl ring of the phenyl-X-phenyl moiety at the ortho-, meta- or para-position relative to X. Preferably, the amine component is attached, through the methylene moiety, at the ortho- or meta-position. More preferably, the amine component is attached, through the methylene moiety, at the meta-position.

For the aminoalkyl-substituted aryl compounds of Formula I, each R₁ subtituent may be positioned ortho-, meta- or para-, relative to X. Preferably, R₁ is positioned at the meta- or para-position. More preferably, R₁ is positioned at the meta-position.

The term “alkyl” as employed herein by itself or as part of another group refers to both straight and branched chain radicals having 1 to 10 carbon atoms, unless the chain length is otherwise specified, including, but not limited to, methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, and the like. Preferred alkyl groups include those having 1 to 6 carbon atoms.

The term “alkenyl” is used herein to mean a straight or branched chain radical of 2-10 carbon atoms, unless the chain length is otherwise specified, wherein there is at least one double bond between two of the carbon atoms in the chain, including, but not limited to, ethenyl, 1-propenyl, 2-propenyl, 2-methyl-1-propenyl, 1-butenyl, 2-butenyl, and the like. Preferably, the alkenyl chain is 2 to 8 carbon atoms in length, more preferably from 2 to 4 carbon atoms in length.

The term “alkynyl” is used herein to mean a straight or branched chain radical of 2-10 carbon atoms, unless the chain length is otherwise specified, wherein there is at least one triple bond between two of the carbon atoms in the chain, including, but not limited to, ethynyl, 1-propynyl, 2-propynyl, and the like. Preferably, the alkynyl chain is 2 to 8 carbon atoms in length, more preferably from 2 to 4 carbon atoms in length.

In all instances herein where there is an alkenyl or alkynyl moiety as a substituent group, the unsaturated linkage, i.e., the vinyl or ethenyl linkage, is preferably not directly attached to a nitrogen, oxygen or sulfur moiety.

The term “alkoxy” or “alkyloxy” refers to any of the above alkyl groups linked to an oxygen atom. Typical examples include methoxy, ethoxy, isopropyloxy, sec-butyloxy and t-butyloxy.

The term “aryl” as employed herein by itself or as part of another group means a C₆₋₁₄ mono- or polycyclic aromatic ring system. Preferably the ring system contains 6 to 10 carbon atoms. Typical examples include phenyl, naphthyl, phenanthryl, anthracyl, indenyl, azulenyl, biphenyl, biphenylenyl and fluorenyl groups. Particularly useful carbocyclic aryl groups include phenyl and naphthyl.

The term “aralkyl” or “arylalkyl” as employed herein by itself or as part of another group refers to C₁₋₆ alkyl groups as discussed above having an aryl substituent, including, but not limited to, benzyl, phenylethyl or 2-naphthylmethyl.

The term “heteroaryl” as employed herein refers to groups having 5 to 14 ring atoms; sharing 6, 10 or 14 pi electrons in a cyclic array; and containing carbon atoms and 1, 2, 3 or 4 heteroatoms independently selected from oxygen, nitrogen and sulfur. Examples of heteroaryl groups include thienyl, benzo[b]thienyl, naphtho[2,3-b]thienyl, thianthrenyl, furanyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4αH-carbazolyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl and tetrazolyl groups.

The term “heterocycle” as employed herein, by itself or as part of another group, refers to a saturated or partially unsaturated ring system having to 14 ring atoms selected from carbon atoms and 1, 2, 3 or 4 heteroatoms independently selected from oxygen, nitrogen and sulfur. Typical examples of saturated heterocycles include pyrrolidinyl, imidazolidinyl, pyrazolidinyl, tetrahydrofuranyl, tetrahydropyranyl, piperidyl, piperazinyl, quinuclidinyl, morpholinyl and dioxacyclohexyl. Typical examples of partially unsaturated heterocycles include pyrrolinyl, imidazolinyl, pyrazolinyl, dihydropyridinyl, tetrahydropyridinyl, and dihydropyranyl. Each of these systems is optionally fused to a benzene ring.

The terms “heteroarylalkyl” or “heteroaralkyl” as employed herein both refer to a heteroaryl group attached to a C₁₋₆ alkyl group. Typical examples include 2-(3-pyridyl)ethyl, 3-(2-furyl)-n-propyl, 3-(3-thienyl)-n-propyl and 4-(1-isoquinolinyl)-n-butyl.

The term “cycloalkyl” as employed herein by itself or as part of another group refers to cycloalkyl groups containing 3 to 9 carbon atoms, unless the size is otherwise specified. Typical examples include cyclopropyl, cyclobutyl, cyclopentyl, cyclohexyl, cycloheptyl and cyclooctyl.

The term “halogen” or “halo” as employed herein by itself or as part of another group refers to chlorine, bromine, fluorine or iodine.

The term “monoalkylamine” or “monoalkylamino” as employed herein by itself or as part of another group refers to the group NH₂ wherein one hydrogen has been replaced by an alkyl group, as defined above.

The term “dialkylamine” or “dialkylamino” as employed herein by itself or as part of another group refers to the group NH₂ wherein both hydrogens have been replaced by alkyl groups, as defined above.

The term “hydroxyalkyl” as employed herein refers to any of the above alkyl groups wherein one or more hydrogens thereof are replaced with one or more hydroxyl moieties.

The term “haloalkyl” as employed herein refers to any of the above alkyl groups wherein one or more hydrogens thereof are substituted by one or more halo moieties. Typical examples include fluoromethyl, difluoromethyl, trifluoromethyl, trichloroethyl, trifluoroethyl, fluoropropyl and bromobutyl.

The term “optionally substituted,” when not further defined, means optional replacement of one or more carbon-attached hydrogens with halogen, halo(C₁₋₆)alkyl, aryl, heterocycle, cycloalkyl, C₁₋₆ alkyl, C₂₋₆ alkenyl, C₂₋₆ alkynyl, aryl(C₁₋₆)alkyl, aryl(C₂₋₆)alkenyl, aryl(C₂₋₆)alkynyl, cycloalkyl(C₁₋₆) alkyl, heterocyclo(C₁₋₆ alkyl), hydroxy(C₁₋₆)alkyl, amino(C₁₋₆)alkyl, carboxy(C₁₋₆)alkyl, alkyloxy(C₁₋₆)alkyl, nitro, amino, ureido, cyano, acylamino, hydroxy, thiol, acyloxy, azido, alkyloxy, carboxy, aminocarbonyl and C₁₋₆ alkylthiol. Preferred optional substituents on a linear carbon chain, when not otherwise specified, include halogen, hydroxy, alkoxy, cyano, amino, nitro, aryl, heteroaryl and heterocycle. Preferred “optionally-substituted alkyl” include C₁₋₆ alkyl, C₁₋₆ haloalkyl, C₁₋₆ hydroxyalkyl and C₁₋₆ alkyloxy(C₁₋₆)alkyl. Preferred optional substituents on a carbon atom that is part of a ring system, when not otherwise specified, include halogen, hydroxy, alkoxy, cyano, amino, nitro, aryl, heteroaryl, heterocycle and alkyl. More preferred optional substituents on a carbon atom that is part of a ring system, when not otherwise specified, include halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and, amino.

Preferred values of n include 1 and 2. A more preferred value of n is 1.

Preferably, R₁ is positioned meta or para relative to X. More preferably, R₁ is positioned meta relative to X.

Preferred R₁ include halogen, C₁₋₆ alkyl and C₁₋₆ haloalkyl. More prefered R₁ include C₁₋₆ haloalkyl. Useful R₁ include trifluoromethyl.

Preferred X include —O—, —S—, —CH₂—O— and —CH₂—S—. More preferred X include —O— and —S—. Most preferred X include —O—.

Preferably, the aminoalkyl moiety (i.e., —CH₂—NR₂R₃) is positioned ortho or meta relative to X. More preferably, the aminoalkyl moiety is positioned meta relative to X.

Preferred values of m include 2 and 3. A more preferred value of m is 2.

Preferred R₄ and R₅ include R₄ and R₅ that together with the nitrogen to which they are attached form a ring having 4 or 5 carbon atoms, which ring optionally contains 1 or 2 additional heteroatoms independently selected from —O—, —S— and —NR₆—, wherein R₆ is hydrogen, C₁₋₆ alkyl, C₁₋₆ haloalkyl or C₁₋₆ hydroxyalkyl. More preferred R₄ and R₅ include R₄ and R₅ that together with the nitrogen to which they are attached form a ring having 4 or 5 carbon atoms, which ring optionally contains 1 additional heteroatom selected from —O—, —S—, and —NR₆—, wherein R₆ is hydrogen or C₁₋₆ alkyl. Most preferred R₄ and R₅ include R₄ and R₅ that together with the nitrogen to which they are attached form a ring having 4 or 5 carbon atoms, preferably 5 carbon atoms.

Preferred R₄ and R₅ also include hydrogen and optionally-substituted C₁₋₆ alkyl. More preferred R₄ and R₅ also include hydrogen, C₁₋₆ alkyl and C₁₋₆ haloalkyl. Most preferred R₄ and R₅ also include hydrogen and C₁₋₆ alkyl.

When R₃ is hydrogen or optionally-substituted C₁₋₆ alkyl, preferred R₃ include hydrogen, C₁₋₆ alkyl, C₁₋₆ haloalkyl, C₁₋₆ hydroxyalkyl and C₁₋₆ alkyloxy(C₁₋₆)alkyl. More preferred R₃ include hydrogen and C₁₋₆ alkyl.

Useful R₃ when R₃ is hydrogen or optionally-substituted C₁₋₆ alkyl include hydrogen.

When R₃ is —(C═R₇)—R₈, preferred R₇ include oxygen.

When R₃ is —(C═R₇)—R₈, preferred R₈ include optionally-substituted C₁₋₆ alkyl, optionally-substituted C₃₋₈ cycloalkyl and optionally-substituted C₆₋₁₀ aryl. More preferred R₈ include C₁₋₆ alkyl, C₁₋₆ haloalkyl, C₁₋₆ hydroxyalkyl, optionally-substituted C₅₋₆ cycloalkyl and optionally-substituted phenyl. Most preferred R₈ include C₁₋₆ alkyl, C₅₋₆ cycloalkyl and optionally-substituted phenyl.

When R₈ is optionally-substituted C₃₋₈ cycloalkyl or optionally-substituted C₆₋₁₀ aryl, it is substituted preferably zero, 1 or 2 times, more preferably zero or one time; and each occurrence of substitution is independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl.

Useful R₃ when R₃ is —(C═R₇)—R₈ include acetyl, cyclopentanecarbonyl and p-fluorobenzoyl.

When R₃ is —(C═R₇)-Z-(CH₂)ₚ—R₉, preferred R₇ include oxygen

When R₃ is —(C═R₇)-Z-(CH₂)ₚ—R₉, preferred Z include —NH—.

When R₃ is —(C═R₇)-Z-(CH₂)ₚ—R₉, preferred values of p include zero, 1, 2 and 3. A preferred value of p is zero. Another preferred value of p is 3.

When R₃ is —(C═R₇)-Z-(CH₂)ₚ—R₉, preferred R₉ include optionally-substituted C₁₋₆ alkyl, optionally-substituted C₃₋₈ cycloalkyl, optionally-substituted C₆₋₁₀ aryl, optionally-substituted heteroaryl and optionally-substituted saturated or partially unsaturated heterocycle. More preferred R₉ include C₁₋₆ alkyl, C₁₋₆ haloalkyl, C₁₋₆ hydroxyalkyl, optionally-substituted C₅₋₆ cycloalkyl, optionally-substituted phenyl and optionally-substituted 5- to 6-membered saturated or partially unsaturated heterocycle. Most preferred R₉ include C₅₋₆ cycloalkyl, optionally-substituted phenyl and 5- to 6-membered saturated or partially unsaturated heterocycle.

When R₉ is optionally-substituted cycloalkyl, optionally-substituted aryl, optionally-substituted heteroaryl or optionally-substituted saturated or partially unsaturated heterocycle, it is substituted preferably zero, 1 or 2 times, more preferably zero or one time; and each occurrence of substitution preferably is independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl-

Useful heteroaryl groups include pyridyl, carbazolyl, furanyl and imidazolyl.

Useful heterocycles include pyrrolidine, piperidine and morpholine.

Useful R₃ when R₃ is —(C═R₇)-Z-(CH₂)ₚ—R₉ include cyclohexylaminocarbonyl, 2-fluorophenylaminocarbonyl and 3-(morpholin-4-yl)-propylaminothiocarbonyl.

In this first aspect of the present invention, preferred compounds of Formula I include those wherein R₃ is hydrogen or optionally-substituted C₁₋₆ alkyl; R₄ and R₅ together with the nitrogen to which they are attached form a ring having 4 or 5 carbon atoms, which ring optionally contains 1 or 2 additional heteroatoms independently selected from —O—, —S— and —NR₆—, wherein R₆ is hydrogen, C₁₋₆ alkyl, C₁₋₆ haloalkyl or C₁₋₆ hydroxyalkyl; X is —O—, —S—, —CH₂—O— or —CH₂—S—; and n, R₁, and m are as defined above. More preferred compounds of Formula I include those wherein R₃ is hydrogen, C₁₋₆ alkyl, C₁₋₆ haloalkyl, C₁₋₆ hydroxyalkyl or C₁₋₆ alkyloxy(C₁₋₆)alkyl; R₄ and R₅ together with the nitrogen to which they are attached form a ring having 4 or 5 carbon atoms, which ring optionally contains 1 or 2 additional heteroatoms selected from —O—, —S—, and —NR₆—, wherein R₆ is hydrogen or C₁₋₆ alkyl, wherein the ring is preferably piperidyl; n is 1 or 2, preferably 1; R₁ is halogen, C₁₋₆ alkyl or C₁₋₆ haloalkyl; X is —O— or —S—; and m is 2 or 3, preferably 2. Particularly preferred compounds of Formula I include those wherein R₃ is hydrogen or C₁₋₆ alkyl; R₄ and R₅ together with the nitrogen to which they are attached form a ring having 4 or 5 carbon atoms, which ring optionally contains 1 additional heteroatom independently selected from —O—, —S— and —NR₆—, wherein R₆ is hydrogen, C₁₋₆ alkyl, C₁₋₆ haloalkyl or C₁₋₆ hydroxyalkyl, wherein the ring is preferably piperidyl; n is 1; R₁ is C₁₋₆ haloalkyl; X is —O—; and m is 2.

In this first aspect of the present invention, preferred compounds of Formula I also include those wherein R₃ is hydrogen or optionally-substituted C₁₋₆ alkyl; R₄ and R₅ are independently selected from hydrogen and optionally-substituted C₁₋₆ alkyl; X is —O—, —S—, —CH₂—O— or —CH₂—S—; and n, R₁, and m are as defined above. More preferred compounds of Formula I also include those wherein R₃ is hydrogen, C₁₋₆ alkyl, C₁₋₆ haloalkyl, C₁₋₆ hydroxyalkyl or C₁₋₆ alkyloxy(C₁₋₆)alkyl; R₄ and R₅ are independently selected from hydrogen, C₁₋₆ alkyl and C₁₋₆ haloalkyl; n is 1 or 2, preferably 1; R₁ is halogen, C₁₋₆ alkyl or C₁₋₆ haloalkyl; X is —O— or —S—; and m is 2 or 3, preferably 2. Particularly preferred compounds of Formula I also include those wherein R₃ is hydrogen or C₁₋₆ alkyl; R₄ and R₅ are independently selected from hydrogen and C₁₋₆ alkyl; n is 1; R₁ is C₁₋₆ haloalkyl; X is —O—; and m is 2.

In this first aspect of the present invention, preferred compounds of Formula I include those wherein R₃ is —(C═R₇)—R₉, wherein R₇ is oxygen or sulfur; R₈ is optionally-substituted C₁₋₆ alkyl, optionally-substituted C₃₋₈ cycloalkyl or optionally-substituted C₆₋₁₀ aryl; R₄ and R₅ together with the nitrogen to which they are attached form a ring having 4 or 5 carbon atoms, which ring optionally contains 1 or 2 additional heteroatoms independently selected from —O—, —S— and —NR₆—, wherein R₆ is hydrogen, C₁₋₆ alkyl, C₁₋₆ haloalkyl or C₁₋₆ hydroxyalkyl; X is —O—, —S—, —CH₂—O— or —CH₂—S—; and n, R₁, and m are as defined above. More preferred compounds of Formula I include those in which R₃ is —(C═R₇)—R₈ wherein R₇ is oxygen; R₈ is C₁₋₆ alkyl, C₁₋₆ haloalkyl, C₁₋₆ hydroxyalkyl, optionally-substituted C₅₋₆ cycloalkyl or optionally-substituted phenyl; R₄ and R₅ together with the nitrogen to which they are attached form a ring having 4 or 5 carbon atoms, which ring optionally contains 1 or 2 additional heteroatoms selected from —O—, —S—, and —NR₆—, wherein R₆ is hydrogen or C₁₋₆ alkyl, wherein the ring is preferably piperidyl; n is 1 or 2, preferably 1; R is halogen, C₁₋₆ alkyl or C₁₋₆ haloalkyl; X is —O— or —S—; and m is 2 or 3, preferably 2. Particularly preferred compounds of Formula I include those in which R₃ is —(C═R₇)—R₈ wherein R₇ is oxygen; R₈ is C₁₋₆ alkyl, C₅₋₆ cycloalkyl or phenyl, wherein the phenyl is substituted with zero, 1 or 2, preferably zero or one, groups independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl; R₄ and R₅ together with the nitrogen to which they are attached form a ring having 4 or 5 carbon atoms, which ring optionally contains 1 additional heteroatom independently selected from —O—, —S— and —NR₆—, wherein R₆ is hydrogen, C₁₋₆ alkyl, C₁₋₆ haloalkyl or C₁₋₆ hydroxyalkyl, wherein the ring is preferably piperidyl; n is 1; R₁ is C₁₋₆ haloalkyl; X is —O—; and m is 2.

In this first aspect of the present invention, preferred compounds of Formula I also include those wherein R₃ is —(C═R₇)—R₈, wherein R₇ is oxygen or sulfur; R₈ is optionally-substituted C₁₋₆ alkyl, optionally-substituted C₃₋₈ cycloalkyl or optionally-substituted C₆₋₁₀ aryl; R₄ and R₅ are independently selected from hydrogen and optionally-substituted C₁₋₆ alkyl; X is —O—, —S—, —CH₂—O— or —CH₂—S—; and n, R₁, and m are as defined above. More preferred compounds of Formula I also include those in which R₃ is —(C═R₇)—R₈ wherein R₇ is oxygen; R₈ is C₁₋₆ alkyl, C₁₋₆ haloalkyl, C₁₋₆ hydroxyalkyl, optionally-substituted C₅₋₆ cycloalkyl or optionally-substituted phenyl; R₄ and R₅ are independently selected from hydrogen, C₁₋₆ alkyl and C₁₋₆ haloalkyl; n is 1 or 2, preferably 1; R₁ is halogen, C₁₋₆ alkyl or C₁₋₆ haloalkyl; X is —O— or —S—; and m is 2 or 3, preferably 2. Particularly preferred compounds of Formula I also include those in which R₃ is —(C═R₇)—R₈ wherein R₇ is oxygen; R₈ is C₁₋₆ alkyl, C₅₋₆ cycloalkyl or phenyl, wherein the phenyl is substituted with zero, 1 or 2, preferably zero or one, groups independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl; R₄ and R₅ are independently selected from hydrogen and C₁₋₆ alkyl; n is 1; R is C₁₋₆ haloalkyl; X is —O—; and m is 2.

In this first aspect of the present invention, preferred compounds of Formula I include those wherein R₃ is —(C═R₇)-Z-(CH₂)ₚ—R₉, wherein R₇, Z and p are defined as above; R₉ is optionally-substituted C₁₋₆ alkyl, optionally-substituted C₃₋₈ cycloalkyl, optionally-substituted C₆₋₁₀ aryl, optionally-substituted heteroaryl or optionally-substituted saturated or partially unsaturated heterocycle; R₄ and R₅ together with the nitrogen to which they are attached form a ring having 4 or 5 carbon atoms, which ring optionally contains 1 or 2 additional heteroatoms independently selected from —O—, —S— and —NR₆—, wherein R₆ is hydrogen, C₁₋₆ alkyl, C₁₋₆ haloalkyl or C₁₋₆ hydroxyalkyl; X is —O—, —S—, —CH₂—O— or —CH₂—S—; and n, R₁, and m are as defined above. More preferred compounds of Formula I include those wherein R₃ is —(C═R₇)-Z-(CH₂)ₚ—R₉, wherein R₇ is oxygen; Z is —NH—; p is zero, 1, 2 or 3; R₉ is C₁₋₆ alkyl, C₁₋₆ haloalkyl, C₁₋₆ hydroxyalkyl, optionally-substituted C₅₋₆ cycloalkyl, optionally-substituted phenyl or optionally-substituted 5- to 6-membered saturated or partially unsaturated heterocycle; R₄ and R₅ together with the nitrogen to which they are attached form a ring having 4 or 5 carbon atoms, which ring optionally contains 1 or 2 additional heteroatoms selected from —O—, —S—, and —NR₆—, wherein R₆ is hydrogen or C₁₋₆ alkyl, wherein the ring is preferably piperidyl; n is 1 or 2, preferably 1; R₁ is halogen, C₁₋₆ alkyl or C₁₋₆ haloalkyl; X is —O— or —S—; and m is 2 or 3, preferably 2. Particularly preferred compounds of Formula I include those wherein R₃ is —(C═R₇)-Z-(CH₂)ₚ—R₉, wherein R₇ is oxygen; Z is —NH—; p is zero, 1, 2 or 3; R₉ is C₅₋₆ cycloalkyl, optionally-substituted phenyl or 5- to 6-membered saturated or partially unsaturated heterocycle, wherein the phenyl is substituted with zero, 1 or 2, preferably zero or one, groups independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl; R₄ and R₅ together with the nitrogen to which they are attached form a ring having 4 or 5 carbon atoms, which ring optionally contains 1 additional heteroatom independently selected from —O—, —S— and —NR₆—, wherein R₆ is hydrogen, C₁₋₆ alkyl, C₁₋₆ haloalkyl or C₁₋₆ hydroxyalkyl, wherein the ring is preferably piperidyl; n is 1; R₁ is C₁₋₆ haloalkyl; X is —O—; and m is 2.

In this first aspect of the present invention, preferred compounds of Formula I also include those wherein R₃ is —(C═R₇)-Z-(CH₂)ₚ—R₉, wherein R₇, Z and p are defined as above; R₉ is optionally-substituted C₁₋₆ alkyl, optionally-substituted C₃₋₈ cycloalkyl, optionally-substituted C₆₋₁₀ aryl, optionally-substituted heteroaryl or optionally-substituted saturated or partially unsaturated heterocycle; R₄ and R₅ are independently selected from hydrogen and optionally-substituted C₁₋₆ alkyl; X is —O—, —S—, —CH₂—O— or —CH₂—S—; and n, R₁, and m are as defined above. More preferred compounds of Formula I also include those wherein R₃ is —(C═R₇)-Z-(CH₂)ₚ—R₉, wherein R₇ is oxygen; Z is —NH—; p is zero, 1, 2 or 3; R₉ is C₁₋₆ alkyl, C₁₋₆ haloalkyl, C₁₋₆ hydroxyalkyl, optionally-substituted C₅₋₆ cycloalkyl, optionally-substituted phenyl or optionally-substituted 5- to 6-membered saturated or partially unsaturated heterocycle; R₄ and R₅ are independently selected from hydrogen, C₁₋₆ alkyl and C₁₋₆ haloalkyl; n is 1 or 2, preferably 1; R₁ is halogen, C₁₋₆ alkyl or C₁₋₆ haloalkyl; X is —O— or —S—; and m is 2 or 3, preferably 2. Particularly preferred compounds of Formula I also include those wherein R₃ is —(C═R₇)-Z-(CH₂)ₚ—R₉, wherein R₇ is oxygen; Z is —NH—; p is zero, 1, 2 or 3; R₉ is C₅₋₆ cycloalkyl, optionally-substituted phenyl or 5- to 6-membered saturated or partially unsaturated heterocycle, wherein the phenyl is substituted with zero, 1 or 2, preferably zero or one, groups independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl; R₄ and R₅ are independently selected from hydrogen and C₁₋₆ alkyl; n is 1; R₁ is C₁₋₆ haloalkyl; X is —O—; and m is 2.

Exemplary preferred compounds that can be employed in this method of the invention include, without limitation:
N-(2-piperidin-1-ylethyl)-N-[3-(3-trifluoromethylphenoxy)benzyl]-amine;
N-(2-piperidin-1-ylethyl)-N-[3-(3-trifluoromethylphenoxy)benzyl]-acetamide;
N-(2-piperidin-1-ylethyl)-N-[3-(3-trifluoromethylphenoxy)benzyl]-cyclopentane carboxamide;
N-(2-piperidin-1-ylethyl)-N-[3-(3-trifluoromethylphenoxy)benzyl]-4-fluorobenzamide;
N′-cyclohexyl-N-(2-piperidin-1-ylethyl)-N-[3-(3-trifluoromethyl-phenoxy)benzyl]urea;
N′-(2-fluorophenyl)-N-(2-piperidin-1-ylethyl)-N-[3-(3-trifluoro-methylphenoxy)benzyl]urea; and
N′-[3-(morpholin-4-yl)propyl]-N-(2-piperidin-1-ylethyl)-N-[3-(3-trifluoromethylphenoxy)benzyl]thiourea;
   and pharmaceutically-acceptable salts thereof.

Particularly preferred compounds include
N-(2-piperidin-1-ylethyl)-N-[3-(3-trifluoromethylphenoxy)benzyl]-cyclopentane carboxamide;
N-(2-piperidin-1-ylethyl)-N-[3-(3-trifluoromethylphenoxy)benzyl]-4-fluorobenzamide;
N′-cyclohexyl-N-(2-piperidin-1-ylethyl)-N-[3-(3-trifluoromethyl-phenoxy)benzyl]urea; and
N′-(2-fluorophenyl)-N-(2-piperidin-1-ylethyl)-N-[3-(3-trifluoromethyl-phenoxy)benzyl]urea;
   and pharmaceutically-acceptable salts thereof.

A second aspect of the present invention is directed to novel compounds used in the method of the first aspect of the present invention and in pharmaceutical compositions thereof.

The novel aminoalkyl-substituted aryl compounds of the second aspect of the present invention are represented by Formula I: 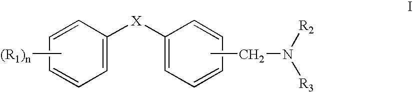
or a pharmaceutically-acceptable salt or solvate thereof, wherein:
R₁ is at each occurrence selected from halogen, optionally-substituted C₁₋₆ alkyl, amino, nitro and cyano;
n is an integer from 1 to 3;
X is —O—, —S—, —NH—, —NHCH₂—, —CH₂NH—, —CH₂—, —CH₂O—, —OCH₂—, —CH₂S— or —SCH₂—;
R₂ is 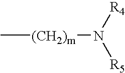
   wherein:
m is an integer from 2 to 4;
R₄ and R₅ are independently selected from hydrogen and optionally-substituted C₁₋₆ alkyl; or R₄ and R₅ together with the nitrogen to which they are attached form a ring having 4 or 5 carbon atoms, which ring optionally contains 1 or 2 additional heteroatoms independently selected from —O—, —S—, and —NR₆—, wherein R₆ is hydrogen, C₁₋₆ alkyl, C₁₋₆ haloalkyl or C₁₋₆ hydroxyalkyl; and
R₃ is hydrogen, optionally-substituted C₁₋₆ alkyl, 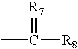
   wherein:
R₇ is oxygen or sulfur; and
R₈ is selected from optionally-substituted C₁₋₆ alkyl, an optionally-substituted C₃₋₈ carbocyclic ring system and optionally-substituted C₆₋₁₀ aryl, or R₃ is 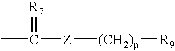
   wherein:
R₇ is oxygen or sulfur;
Z is —O— or —NH—;
p is an integer from 0 to 4; and
R₉ is selected from optionally-substituted C₁₋₆ alkyl, an optionally-substituted C₃₋₈ carbocyclic ring system, optionally-substituted C₆₋₁₀ aryl, optionally-substituted heteroaryl and optionally-substituted heterocycle, wherein the heterocycle is saturated or partially unsaturated; provided that,
when R₄ and R₅ are independently hydrogen or C₁₋₂ alkyl, or when R₄ and R₅ together with the nitrogen to which they are attached form pyrrolidinyl, then X is not —S—;
when X is —CH₂O— and R₃ is hydrogen or methyl, then at least one of R₄ or R₅ is not C₃₋₅ alkyl; and
when X is —O— and R₄ and R₅ together with the nitrogen to which they are attached form a ring having 4 or 5 carbon atoms, then R₈ is not a C₃ carbocyclic ring system, and R₉ is not C₃₋₅ alkyl, phenyl, dihalophenyl or (C₁₋₂ alkyl)phenyl.

The particulars and preferences of each of the substituents, regarding both their identities and values and their attachment positions, which were discussed above for the first aspect of the present invention, apply also to the second aspect of the present invention.

In one embodiment, compounds in the second aspect of the invention are those preferred in the first aspect of the present invention, as discussed above, with the additional preferences that:
when X is oxygen, R₃ is —(C═R₇)R₈, and R₄ and R₅ together with the nitrogen to which they are attached form a ring having 4 or 5 carbon atoms, which ring optionally contains 1 or 2 additional heteroatoms independently selected from —O—, —S—, and —NR₆—, then:

R₈ is preferably selected from C₁₋₆ alkyl, C₁₋₆ haloalkyl, C₁₋₆ hydroxyalkyl, C₁₋₆ alkyloxyalkyl, optionally-substituted C₅₋₈ cycloalkyl, and C₆₋₁₀ aryl, wherein said C₆₋₁₀ aryl is optionally substituted with halogen, C₁₋₆ alkyl, C₁₋₆ haloalkyl, or C₁₋₆ hydroxyalkyl; more preferably selected from C₁₋₆ alkyl, C₅₋₆ cycloalkyl, and C₆₋₁₀ aryl, wherein said C₆₋₁₀ aryl is optionally substituted with halogen;
when X is oxygen, R₃ is —(C═R₇)-Z-(CH₂)ₚ—R₉, and R₄ and R₅ together with the nitrogen to which they are attached form a ring having 4 or 5 carbon atoms, which ring optionally contains 1 or 2 additional heteroatoms independently selected from —O—, —S—, and —NR₆—, then:
R₉ is preferably selected from the group consisting of optionally-substituted C₁₋₂ alkyl, optionally-substituted C₆ alkyl, optionally-substituted C₃₋₈ cycloalkyl, heteroaryl, heterocycle, wherein said heterocycle is saturated or partially saturated, optionally substituted C₇₋₁₀ aryl, and phenyl substituted with one or more of C₃₋₆ alkyl, C₁₋₆ haloalkyl, C₁₋₆ hydroxyalkyl, and C₁₋₆ alkyloxyalkyl, or substituted once with halogen; more preferably selected from the group consisting of C₅₋₆ cycloalkyl and heterocycle, wherein said heterocycle is saturated or partially unsaturated; and
when R₄ and R₅ are independently hydrogen or C₁₋₂ alkyl, then:
X is preferably —O—, —CH₂—O—, or —CH₂—S—; more preferably —O—.

In this second aspect of the present invention, preferred compounds of Formula I include those wherein R₃ is hydrogen or optionally-substituted C₁₋₆ alkyl; R₄ and R₅ together with the nitrogen to which they are attached form a ring having 4 or 5 carbon atoms, which ring optionally contains 1 or 2 additional heteroatoms independently selected from —O—, —S— and —NR₆—, wherein R₆ is hydrogen, C₁₋₆ alkyl, C₁₋₆ haloalkyl or C₁₋₆ hydroxyalkyl; X is —O—, —CH₂—O— or —CH₂—S—; and n, R₁, and m are as defined above. More preferred compounds of Formula I include those wherein R₃ is hydrogen, C₁₋₆ alkyl, C₁₋₆ haloalkyl, C₁₋₆ hydroxyalkyl or C₁₋₆ alkyloxy(C₁₋₆)alkyl; R₄ and R₅ together with the nitrogen to which they are attached form a ring having 4 or 5 carbon atoms, which ring optionally contains 1 or 2 additional heteroatoms selected from —O—, —S—, and —NR₆—, wherein R₆ is hydrogen or C₁₋₆ alkyl, wherein the ring is preferably piperidyl; n is 1 or 2, preferably 1; R₁ is halogen, C₁₋₆ alkyl or C₁₋₆ haloalkyl; X is —O— or —CH₂—O—; and m is 2 or 3, preferably 2. Particularly preferred compounds of Formula I include those wherein R₃ is hydrogen or C₁₋₆ alkyl; R₄ and R₅ together with the nitrogen to which they are attached form a ring having 4 or 5 carbon atoms, which ring optionally contains 1 additional heteroatom independently selected from —O—, —S— and —NR₆—, wherein R₆ is hydrogen, C₁₋₆ alkyl, C₁₋₆ haloalkyl or C₁₋₆ hydroxyalkyl, wherein the ring is preferably piperidyl; n is 1; R₁ is C₁₋₆ haloalkyl; X is —O—; and m is 2.

In this second aspect of the present invention, preferred compounds of Formula I also include those wherein R₃ is hydrogen or optionally-substituted C₁₋₆ alkyl; one of R₄ or R₅ is selected from hydrogen and optionally-substituted C₁₋₆ alkyl, and the other is selected from hydrogen, optionally-substituted C₁₋₂ alkyl and optionally-substituted C₁₋₆ alkyl; X is —O—, —CH₂—O— or —CH₂—S—; and n, R₁, and m are as defined above. More preferred compounds of Formula I also include those wherein R₃ is hydrogen, C₁₋₆ alkyl, C₁₋₆ haloalkyl, C₁₋₆ hydroxyalkyl or C₁₋₆ alkyloxy(C₁₋₆)alkyl; one of R₄ or R₅ is selected from hydrogen, C₁₋₆ alkyl and C₁₋₆ haloalkyl, and the other is selected from hydrogen, C₁₋₂ alkyl, C₆ alkyl, C₁₋₂ haloalkyl and C₆ haloalkyl; n is 1 or 2, preferably 1; R₁ is halogen, C₁₋₆ alkyl or C₁₋₆ haloalkyl; X is —O— or —CH₂—O— and m is 2 or 3, preferably 2. Particularly preferred compounds of Formula I also include those wherein R₃ is hydrogen or C₁₋₆ alkyl; R₄ and R₅ are independently selected from hydrogen and C₁₋₆ alkyl; n is 1; R₁ is C₁₋₆ haloalkyl; X is —O—; and m is 2.

In this second aspect of the present invention, preferred compounds of Formula I include those wherein R₃ is —(C═R₇)—R₈, wherein R₇ is oxygen or sulfur; R₈ is optionally-substituted C₁₋₆ alkyl, optionally-substituted C₅₋₈ cycloalkyl or optionally-substituted C₆₋₁₀ aryl; R₄ and R₅ together with the nitrogen to which they are attached form a ring having 4 or 5 carbon atoms, which ring optionally contains 1 or 2 additional heteroatoms independently selected from —O—, —S— and —NR₆—, wherein R₆ is hydrogen, C₁₋₆ alkyl, C₁₋₆ haloalkyl or C₁₋₆ hydroxyalkyl; X is —O—, —CH₂—O— or —CH₂—S—; and n, R₁, and m are as defined above. More preferred compounds of Formula I include those in which R₃ is —(C═R₇)—R₈ wherein R₇ is oxygen; R₉ is C₁₋₆ alkyl, C₁₋₆ haloalkyl, C₁₋₆ hydroxyalkyl, optionally-substituted C₅₋₆ cycloalkyl or optionally-substituted phenyl; R₄ and R₅ together with the nitrogen to which they are attached form a ring having 4 or 5 carbon atoms, which ring optionally contains 1 or 2 additional heteroatoms selected from —O—, —S—, and —NR₆—, wherein R₆ is hydrogen or C₁₋₆ alkyl, wherein the ring is preferably piperidyl; n is 1 or 2, preferably 1; R₁ is halogen, C₁₋₆ alkyl or C₁₋₆ haloalkyl; X is —O— or —CH₂—O—; and m is 2 or 3, preferably 2. Particularly preferred compounds of Formula I include those in which R₃ is —(C═R₇)—R₈ wherein R₇ is oxygen; R₈ is C₁₋₆ alkyl, C₅₋₆ cycloalkyl or phenyl, wherein the phenyl is substituted with zero, 1 or 2, preferably zero or one, groups independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl; R₄ and R₅ together with the nitrogen to which they are attached form a ring having 4 or 5 carbon atoms, which ring optionally contains 1 additional heteroatom independently selected from —O—, —S— and —NR₆—, wherein R₆ is hydrogen, C₁₋₆ alkyl, C₁₋₆ haloalkyl or C₁₋₆ hydroxyalkyl, wherein the ring is preferably piperidyl; n is 1; R₁ is C₁₋₆ haloalkyl; X is —O—; and m is 2.

In this second aspect of the present invention, preferred compounds of Formula I also include those wherein R₃ is —(C═R₇)—R₈, wherein R₇ is oxygen or sulfur; R₈ is optionally-substituted C₁₋₆ alkyl, optionally-substituted C₃₋₈ cycloalkyl or optionally-substituted C₆₋₁₀ aryl; R₄ and R₅ are independently selected from hydrogen and optionally-substituted C₁₋₆ alkyl; X is —O—, —CH₂—O— or —CH₂—S—; and n, R₁, and mare as defined above. More preferred compounds of Formula I also include those in which R₃ is —(C═R₇)—R₈ wherein R₇ is oxygen; R₈ is C₁₋₆ alkyl, C₁₋₆ haloalkyl, C₁₋₆ hydroxyalkyl, optionally-substituted C₅₋₆ cycloalkyl or optionally-substituted phenyl; R₄ and R₅ are independently selected from hydrogen, C₁₋₆ alkyl and C₁₋₆ haloalkyl; n is 1 or 2, preferably 1; R₁ is halogen, C₁₋₆ alkyl or C₁₋₆ haloalkyl; X is —O— or —CH₂—O—; and m is 2 or 3, preferably 2. Particularly preferred compounds of Formula I also include those in which R₃ is —(C═R₇)—R₉ wherein R₇ is oxygen; R₈ is C₁₋₆ alkyl, C₅₋₆ cycloalkyl or phenyl, wherein the phenyl is substituted with zero, 1 or 2, preferably zero or one, groups independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl; R₄ and R₅ are independently selected from hydrogen and C₁₋₆ alkyl; n is 1; R₁ is C₁₋₆ haloalkyl; X is —O—; and m is 2.

In this second aspect of the present invention, preferred compounds of Formula I include those wherein R₃ is —(C═R₇)-Z-(CH₂)ₚ—R₉, wherein R₇, Z and p are defined as above; R₉ is optionally-substituted C₁₋₂ alkyl, optionally-substituted C₆ alkyl, optionally-substituted C₃₋₈ cycloalkyl, substituted C₆₋₁₀ aryl, optionally-substituted heteroaryl or optionally-substituted saturated or partially unsaturated heterocycle; R₄ and R₅ together with the nitrogen to which they are attached form a ring having 4 or 5 carbon atoms, which ring optionally contains 1 or 2 additional heteroatoms independently selected from —O—, —S— and —NR₆—, wherein R₆ is hydrogen, C₁₋₆ alkyl, C₁₋₆ haloalkyl or C₁₋₆ hydroxyalkyl; X is —O—, —CH₂—O— or —CH₂—S—; and n, R₁, and m are as defined above. More preferred compounds of Formula I include those wherein R₃ is —(C═R₇)-Z-(CH₂)ₚ—R₉, wherein R₇ is oxygen; Z is —NH—; p is zero, 1, 2 or 3; R₉ is C₁₋₂ alkyl, C₆ alkyl, C₁₋₆ haloalkyl, C₁₋₆ hydroxyalkyl, optionally-substituted C₅₋₆ cycloalkyl, substituted phenyl or optionally-substituted 5- to 6-membered saturated or partially unsaturated heterocycle; R₄ and R₅ together with the nitrogen to which they are attached form a ring having 4 or 5 carbon atoms, which ring optionally contains 1 or 2 additional heteroatoms select3ed from —O —, —S—, and —NR₆—, wherein R₆ is hydrogen or C₁₋₆ alkyl, wherein the ring is preferably piperidyl; n is 1 or 2, preferably 1; R₁ is halogen, C₁₋₆ alkyl or C₁₋₆ haloalkyl; X is —O— or —CH₂—O—; and m is 2 or 3, preferably 2. Particularly preferred compounds of Formula I include those wherein R₃ is —(C═R₇)-Z-(CH₂)ₚ—R₉, wherein R₇ is oxygen; Z is —NH—; p is zero, 1, 2 or 3; R₉ is C₅₋₆ cycloalkyl, substituted phenyl or 5- to 6-membered saturated or partially unsaturated heterocycle, where the substituted phenyl is phenyl substituted with 1 or 2, preferably one, groups independently selected from halogen, C₃₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₃₋₄ alkyl, C₁₋₄ haloalkyl and C₁₋₄ hydroxyalkyl, most preferably selected from halogen and C₃₋₄ alkyl, and where the substituted phenyl is not dihalophenyl; R₄ and R₅ together with the nitrogen to which they are attached form a ring having 4 or 5 carbon atoms, which ring optionally contains 1 additional heteroatom independently selected from —O—, —S— and —NR₆—, wherein R₆ is hydrogen, C₁₋₆ alkyl, C₁₋₆ haloalkyl or C₁₋₆ hydroxyalkyl, wherein the ring is preferably piperidyl; n is 1; R₁ is C₁₋₆ haloalkyl; X is —O—; and m is 2.

In this second aspect of the present invention, preferred compounds of Formula I also include those wherein R₃ is —(C═R₇)-Z-(CH₂)ₚ—R₉, wherein R₇, Z and p are defined as above; R₉ is optionally-substituted C₁₋₆ alkyl, optionally-substituted C₃₋₈ cycloalkyl, optionally-substituted C₆₋₁₀ aryl, optionally-substituted heteroaryl or optionally-substituted saturated or partially unsaturated heterocycle; R₄ and R₅ are independently selected from hydrogen and optionally-substituted C₁₋₆ alkyl; X is —O—, —CH₂—O— or —CH₂—S—; and n, R₁, and m are as defined above. More preferred compounds of Formula I also include those wherein R₃ is —(C═R₇)-Z-(CH₂)ₚ—R₉, wherein R₇ is oxygen; Z is —NH—; p is zero, 1, 2 or 3; R₉ is C₁₋₆ alkyl, C₁₋₆ haloalkyl, C₁₋₆ hydroxyalkyl, optionally-substituted C₅₋₆ cycloalkyl, optionally-substituted phenyl or optionally-substituted 5- to 6-membered saturated or partially unsaturated heterocycle; R₄ and R₅ are independently selected from hydrogen, C₁₋₆ alkyl and C₁₋₆ haloalkyl; n is 1 or 2, preferably 1; R₁ is halogen, C₁₋₆ alkyl or C₁₋₆ haloalkyl; X is —O— or —CH₂—O—; and m is 2 or 3, preferably 2. Particularly preferred compounds of Formula I also include those wherein R₃ is —(C═R₇)-Z-(CH₂)ₚ—R₉, wherein R₇ is oxygen; Z is —NH—; p is zero, 1, 2 or 3; R₉ is C₅₋₆ cycloalkyl, optionally-substituted phenyl or 5- to 6-membered saturated or partially unsaturated heterocycle, wherein the phenyl is substituted with zero, 1 or 2, preferably zero or one, groups independently selected from halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, more preferably selected from halogen, C₁₋₄ alkyl, C₁₋₄ haloalkyl and C₁₋₄ hydroxyalkyl, most preferably selected from halogen and C₁₋₄ alkyl; R₄ and R₅ are independently selected from hydrogen and C₁₋₆ alkyl; n is 1; R₁ is C₁₋₆ haloalkyl; X is —O—; and m is 2.

In addition to administering the compound as a raw chemical, the compounds of the invention can be administered as part of a pharmaceutical preparation containing suitable pharmaceutically-acceptable carriers comprising excipients and auxiliaries that facilitate processing of the compounds into preparations that can be used pharmaceutically. Preferably, the preparations, particularly those preparations that can be administered orally and that can be used for the preferred type of administration, such as tablets, dragees and capsules, and also preparations that can be administered rectally, such as suppositories, as well as suitable solutions for administration orally or by injection, contain from about 0.01 to 99 percent, preferably from about 0.25 to 75 percent of active compound(s), together with the excipient.

Also included within the scope of the present invention are the non-toxic pharmaceutically-acceptable salts of the compounds of the present invention. Acid addition salts are formed by mixing a solution of a particular aminoalkyl-substituted aryl compound of Formula I, with a solution of a pharmaceutically-acceptable non-toxic acid such as, but not limited to, acetic acid, benzoic acid, carbonic acid, citric acid, dichloroacetic acid, dodecylsulfonic acid, 2-ethylsuccinic acid, fumaric acid, glubionic acid, gluconic acid, hydrobromic acid, hydrochloric acid, 3-hydroxynaphthoic acid, isethionic acid, lactic acid, lactobionic acid, levulinic acid, maleic acid, malic acid, malonic acid, methanesulfic acid, methanesulfonic acid, nitric acid, oxalic acid, phosphoric acid, propionic acid, sulfuric acid, sulfamic acid, saccharic acid, succinic acid, tartaric acid, and the like. Basic amine salts are formed by mixing a solution of the aminoalkyl-substituted aryl compound of the present invention with a solution of a pharmaceutically-acceptable non-toxic acid, such as those listed above, and, preferably, hydrochloric acid or carbonic acid.

The pharmaceutical compositions of the invention can be administered to any animal that can experience the beneficial effects of the compounds of the invention. Foremost among such animals are mammals, e.g., humans, dogs and cats, although the invention is not intended to be so limited.

The pharmaceutical compositions of the present invention can be administered by any means that achieve their intended purpose. For example, administration can be by parenteral, subcutaneous, intravenous, intramuscular, intraperitoneal, transdermal, or buccal routes. Alternatively, or concurrently, administration can be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings that, if desired, are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used. Dye stuffs or pigments can be added to the tablets or dragee coatings, for example, for identification or in order to characterize combinations of active compound doses.

Other oral pharmaceutical preparations include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers can be added.

Possible pharmaceutical preparations, which can be used rectally, include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, and include, for example, sodium carboxymethyl cellulose, sorbitol, and/or dextran. Optionally, the suspension can also contain stabilizers.

A third aspect of the present invention is directed to a method of making the novel aminoalkyl-substituted aryl compounds of Formula I according to the second aspect of the present invention.

The aminoalkyl-substituted aryl compounds of Formula I are prepared by a method comprising reacting, in a first step, an aryl aldehyde with a suitable primary or secondary amine, and, in an optional second step, reacting the product of the first step either (i) with an acid chloride compound of Formula II: 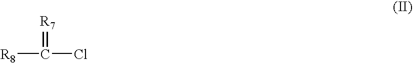
wherein R₇ is oxygen or sulfur, and R₈ is selected from optionally-substituted C₁₋₆ alkyl, an optionally-substituted C₃₋₈ carbocyclic ring system and optionally-substituted C₆₋₁₀ aryl;
or (ii) with an isocyanate of Formula III: 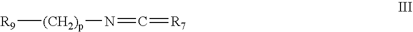
wherein R₇ is oxygen or sulfur, p is an integer from zero to 4, and R₉ is selected from optionally-substituted C₁₋₆ alkyl, an optionally-substituted C₃₋₈ carbocyclic ring system, optionally-substituted C₆₋₁₀ aryl, optionally-substituted heteroaryl and saturated or partially unsaturated heterocycle; and recovering the product obtained in either of the first or second steps. The product obtained from the first step is either a secondary or tertiary amine, while that of the second step is an amide or urea, or a thio analog thereof.

Scheme 1 shows the formation of the secondary amine compounds (i.e., R₃ is hydrogen) and the tertiary amine compounds (i.e., R₃ is alkyl) of Formula I. 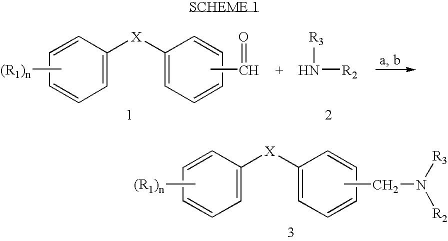
Reagents: (a) titanium (IV) isopropoxide, THF; (b) sodium borohydride, ethanol
Scheme 2 shows the formation of the amide compounds of Formula I (5) and urea compounds of Formula I (7), using compound 3 wherein R₃ is hydrogen (a product of Scheme 1) as a reactant.

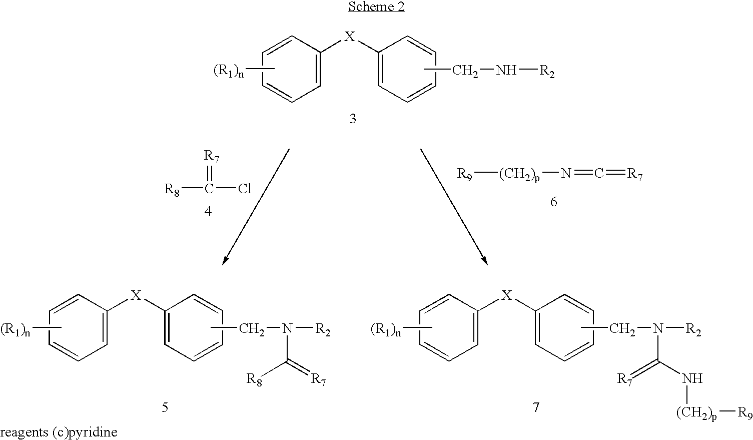

The resulting compounds are purified by flash column chromatography or silica gel chromatography.

The above procedure may also be used to synthesize carbamate and thiocarbamate analogs of compound 7. To form the carbamate, amine 3 is reacted with triphosgene and triethylamine to form an isocyanate. The isocyanate is then reacted with an alcohol of formula R₉—(CH₂)ₚ—OH to form the carbamate. Similarly, the thiocarbamate is formed by reacting amine 3 with thiophosgene and triethylamine to form an isothiocyanate, which is further reacted with an alcohol of formula R₉—(CH₂)ₚ—OH.

The invention disclosed herein is meant to encompass all pharmaceutically-acceptable salts thereof of the disclosed compounds. The pharmaceutically-acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; and amino acid salts such as arginate, asparginate, glutamate and the like.

The invention disclosed herein is also meant to encompass the in vivo metabolic products of the disclosed compounds. Such products can result for example from the oxidation, reduction, hydrolysis, amidation, esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein is also meant to encompass the disclosed compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively.

Some of the compounds disclosed herein may contain one or more asymmetric centers and thus can give rise to enantiomers, diastereomers, and other stereoisomeric forms. The present invention is also meant to encompass all such possible forms as well as their racemic and resolved forms and mixtures thereof. When the compounds described herein contain olefinic double bonds or other centers of geometric asymmetry, and unless specified otherwise, the present invention is intended to include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached, or a sulfur atom to which three different groups are attached, where the sulfur atom and its attached groups form a sulfoxide, sulfinic ester, sulfonium salt or sulfite.

The term “enantiomer” or “enantiomeric” refers to a molecule that is nonsuperimposeable on its mirror image and hence optically active such that the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule. The phrase “enantiomeric excess” refers to a mixture wherein one enantiomer is present is a greater concentration than its mirror image molecule.

The method of the first aspect of the present invention is directed to treating disorders responsive to the blockade of sodium channels in mammals suffering therefrom. Specifically, the method of the present invention utilizing the aminoalkyl-substituted aryl compounds of Formula I can be applied to the treatment of humans or companion animals, such as dogs and cats. Preferred aminoalkyl-substituted aryl compounds of Formula I for use in the method of the first aspect of the present invention are those as defined above.

The effectiveness of the compounds for the method of the present invention is assessed by electrophysiological assays in dissociated hippocampal neurons to determine sodium channel blocker activity. These compounds also are optionally assayed for binding to the neuronal voltage-dependent sodium channel using rat forebrain membranes and [³H]BTX-B.

Sodium channels are large transmembrane proteins that are expressed in various tissues. They are voltage sensitive channels and are responsible for the rapid increase of Na⁺ permeability in response to depolarization associated with the action potential in many excitable cells including muscle, nerve and cardiac cells.

Another aspect of the method of the present invention is the discovery of the mechanism of action of the compounds herein described as specific Na⁺ channel blockers. Based upon the discovery of this mechanism, these compounds are contemplated to be useful in treating or preventing neuronal loss due to focal or global ischemia, and in treating or preventing neurodegenerative disorders including ALS, anxiety, and epilepsy. They are also expected to be effective in treating, preventing or ameliorating neuropathic pain, surgical pain, chronic pain and tinnitus. The compounds are also expected to be useful as antiarrhythmics, anesthetics and antimanic depressants.

The method of the present invention is directed to the use of compounds of Formula I which are blockers of voltage-sensitive sodium channels. According to the present invention, those compounds having preferred sodium channel blocking properties exhibit an IC₅₀ of about 10 μM or less in the electrophysiological assay described herein. Preferably, the compounds of the present invention exhibit an IC₅₀ of 10 μM or less. Most preferably, the compounds of the present invention exhibit an IC₅₀ of about 1.0 μM or less. The following binding and electrophysiological assays can be used to test compounds of the present invention for their Na+ channel blocking activity.

In vitro Binding Assay:

The ability of compounds of the present invention to modulate either site 1 or site 2 of the Na⁺ channel was determined following the procedures fully described in Yasushi, J. Biol. Chem. 261:6149-6152 (1986) and Creveling, Mol. Pharmacol. 23:350-358 (1983), respectively. Rat forebrain membranes are used as sources of Na⁺ channel proteins. The binding assays are conducted in 130 μM choline chloride at 37° C. for 60-minute incubation with [³H] saxitoxin and [³H] batrachotoxin as radioligands for site 1 and site 2, respectively.

In Vivo Pharmacology:

The compounds of the present invention can be tested for in vivo anticonvulsant activity after i.v., p.o. or i.p. injection using a number of anticonvulsant tests in mice, including the maximum electroshock seizure test (MES). Maximum electroshock seizures are induced in male NSA mice weighing between 15-20 g and male Sprague-Dawley rats weighing between 200-225 g by application of current (50 mA, 60 pulses/sec, 0.8 msec pulse width, 1 sec duration, D.C., mice; 99 mA, 125 pulses/sec, 0.8 msec pulse width, 2 sec duration, D.C., rats) using a Ugo Basile ECT device (Model 7801). Mice are restrained by gripping the loose skin on their dorsal surface and saline-coated corneal electrodes are held lightly against the two corneae. Rats are allowed free movement on the bench top and ear-clip electrodes are used. Current is applied and animals are observed for a period of up to 30 seconds for the occurrence of a tonic hindlimb extensor response. A tonic seizure is defined as a hindlimb extension in excess of 90 degrees from the plane of the body. Results are treated in a quantal manner.

The compounds can be tested for their antinociceptive activity in the formalin model as described in Hunskaar, S., O. B. Fasmer, and K. Hole, J. Neurosci. Methods 14: 69-76 (1985). Male Swiss Webster NIH mice (20-30 g; Harlan, San Diego, Calif.) are used in all experiments. Food is withdrawn on the day of experiment. Mice are placed in Plexiglas® jars for at least 1 hour to accommodate to the environment. Following the accommodation period, mice are weighed and given either the compound of interest administered i.p. or p.o., or the appropriate volume of vehicle (10% Tween®-80). Fifteen minutes after the i.p. dosing, and 30 minutes after the p.o. dosing, mice are injected with formalin (20 μL of 5% formaldehyde solution in saline) into the dorsal surface of the right hind paw. Mice are transferred to the Plexiglas® jars and monitored for the amount of time spent licking or biting the injected paw. Periods of licking and biting are recorded in 5 minute intervals for 1 hour after the formalin injection. All experiments are done in a blinded manner during the light cycle. The early phase of the formalin response is measured as licking/biting between 0-5 minutes, and the late phase is measured from 15-50 minutes. Differences between vehicle- and drug-treated groups are analyzed by one-way analysis of variance (ANOVA). A p-value≦0.05 is considered significant. Activity in blocking the acute and second phase of formalin-induced paw-licking activity is indicative that compounds are considered to be efficacious for acute and chronic pain.

The compounds can be tested for their potential for the treatment of chronic pain (antiallodynic and antihyperalgesic activities) in the Chung model of peripheral neuropathy. Male Sprague-Dawley rats weighing between 200-225 g are anesthetized with halothane (1-3% in a mixture of 70% air and 30% oxygen) and their body temperature is controlled during anesthesia through use of a homeothermic blanket. A 2-cm dorsal midline incision is then made at the L5 and L6 level and the para-vertibral muscle groups retracted bilaterally. L5 and L6 spinal nerves are then be exposed, isolated, and tightly ligated with 6-0 silk suture. A sham operation is performed exposing the contralateral L5 and L6 spinal nerves as a negative control.

Tactile Allodynia: Rats are transferred to an elevated testing cage with a wire mesh floor and allowed to acclimate for five to ten minutes. A series of Semmes-Weinstein monofilaments are applied to the plantar surface of the hindpaw to determine the animal's withdrawal threshold. The first filament used possesses a buckling weight of 9.1 g (0.96 log value) and is applied up to five times to see if it elicited a withdrawal response. If the animal has a withdrawal response then the next lightest filament in the series is applied up to five times to determine if it can elicit a response. This procedure is repeated with subsequent less filaments until there is no response and the lightest filament that elicits a response is recorded. If the animal does not have a withdrawal response from the initial 9.1 g filament then subsequent filaments of increased weight are applied until a filament elicits a response and this filament is then recorded. For each animal, three measurements are made at every time point to produce an average withdrawal threshold determination. Tests are performed prior to and at 1, 2, 4 and 24 hours post drug administration. Tactile allodynia and mechanical hyperalgesia tests were conducted concurrently.

Mechanical Hyperalgesia: Rats are transferred to an elevated testing cage with a wire mesh floor and allowed to acclimate for five to ten minutes. A slightly blunted needle is touched to the plantar surface of the hindpaw causing a dimpling of the skin without penetrating the skin. Administration of the needle to control paws typically produces a quick flinching reaction, too short to be timed with a stopwatch and arbitrarily gives a withdrawal time of 0.5 second. The operated side paw of neuropathic animals exhibits an exaggerated withdrawal response to the blunted needle. A maximum withdrawal time of ten seconds is used as a cutoff time. Withdrawal times for both paws of the animals are measured three times at each time point with a five-minute recovery period between applications. The three measures are used to generate an average withdrawal time for each time point. Tactile allodynia and mechanical hyperalgesia tests are conducted concurrently.

The compounds can be tested for their neuroprotective activity after focal and global ischemia produced in rats or gerbils according to the procedures described in Buchan et al., Stroke, Suppl. 148-152 (1993); Sheardown et al., Eur. J. Pharmacol. 236:347-353 (1993); and Graham et al., J. Pharmacol. Exp. Therap. 276:1-4 (1996).

The compounds can be tested for their neuroprotective activity after traumatic spinal cord injury according to the procedures described in Wrathall et al., Exp. Neurology 137:119-126 (1996) and Iwasaki et al., J. Neuro Sci. 134:21-25 (1995).

Electrophysiological Assay:

An electrophysiological assay was used to measure potencies of compounds of the present invention rBIIa/beta 1 sodium channels expressed in Xenopus oocytes.

Preparation of cRNA encoding cloned rat brain type IIa (rBIIa) and beta 1 (β1): cDNA clones encoding the rat brain beta 1 subunit are cloned in house using standard methods, and mRNA are prepared by standard methods. mRNA encoding rBIIa is provided by Dr. A. Golden (UC Irvine). The mRNAs are diluted and stored at −80° C. in 1 μL aliquots until injection.

Preparation of oocytes: Mature female Xenopus laevis are anaesthetized (20-40 min) using 0.15% 3-aminobenzoic acid ethyl ester (MS-222) following established procedures (Woodward, R. M., et al., Mol. Pharmacol. 41:89-103 (1992)).

Two to six ovarian lobes are surgically removed. Oocytes at developmental stages V-VI are dissected from the ovary, wherein the oocytes are still surrounded by enveloping ovarian tissues. Oocytes are defolliculated on the day of surgery by treatment with collagenase (0.5 mg/mL Sigma Type I, or Boehringer Mannheim Type A, for 0.5-1 hr). Treated oocytes are vortexed to dislodge epithelia, washed repeatedly and stored in Barth's medium containing 88 mM NaCl, 1 mM KCl, 0.41 mM CaCl₂, 0.33 mM Ca(NO₃)₂, 0.82 mM MgSO₄, 2.4 mM NaHCO₃, 5 mM HEPES, pH 7.4 adjusted with 0.1 mg/mL gentamycin sulphate.

Micro-injection of oocytes: Defolliculated oocytes are micro-injected using a Nanoject injection system (Drummond Scientific Co., Broomall, Pa.). Injection pipettes are beveled to minimize clogging. Tip diameter of injection pipettes is 15-35 μm. Oocytes are microinjected with approximately 50 nL 1:10 ratio mixtures of cRNAs for rBIIa and beta 1 respectively.

Electrophysiology: Membrane current responses are recorded in frog Ringer solution containing 115 mM NaCl, 2 mM KCl, 1.8 mM CaCl₂, 5 mM HEPES, pH 7.4. Electrical recordings are made using a conventional two-electrode voltage clamp (Dagan TEV-200) over periods ranging between 1-7 days following injection. The recording chamber is a simple gravity fed flow-through chamber (volume 100-500 mL depending on adjustment of aspirator). Oocytes are placed in the recording chamber, impaled with electrodes and continuously perfused (5-15 mL min⁻¹) with frog Ringer's solution. The tested compounds are applied by bath perfusion.

Voltage protocols for evoking sodium channel currents: The standard holding potential for whole oocyte clamp is −120 mV. Standard current-voltage relationships are elicited by 40 ms depolarizing steps starting from −60 mV to +50 mV in 10 mV increments. Peak currents are measured as the maximum negative current after depolarizing voltage steps. The voltage from maximum current response is noted and used for the next voltage protocol.

The purpose is to find compounds that are state dependent modifiers of neuronal sodium channels. Preferably, the compounds have a low affinity for the rested/closed state of the channel, but a high affinity for the inactivated state. The following voltage protocol is used to measure a compounds affinity for the inactivated state. Oocytes are held at a holding potential of −120 mV. At this membrane voltage, nearly all of the channels are in the closed state. Then a 4-second depolarization is made to the voltage where the maximum current is elicited. At the end of this depolarization, nearly all the channels are in the inactivated state. A 10 ms hyperpolarizing step is then made in order to remove some channels from the inactivated state. A final depolarizing test pulse is used to assay the sodium current after this prolonged depolarization (see analysis below). Sodium currents are measured at this test pulse before and after the application of the tested compound. Data is acquired using PCLAMP 8.0 software and analyzed with CLAMPFIT software (Axon instruments).

Data analysis: Apparent inhibition constants (Kᵢ values) for antagonists are determined from single point inhibition data using the following equation (a generalized form of the Cheng-Prusoff equation) (Leff, P. and Dougall, I. G., TiPS 14:110-112 (1993)):
Kᵢ(FR/1−FR)*[drug] Eq.2
where FR is the fractional response and is defined as sodium current elicited from the final depolarizing test pulse prior to application of the drug divided by the sodium current measured in the presence of the drug, and [drug] is the concentration of the drug used.

Drugs: Drugs are initially made up at concentrations of 2-10 mM in DMSO. Dilutions are then made to generate a series of DMSO stocks over the range 0.3 μM to 10 mM, depending upon the potency of the compound. Working solutions are made by 1000- to 3000-fold dilution of stocks into Ringer. At these dilutions DMSO alone has little or no measurable effects on membrane current responses. DMSO stocks of drugs are stored in the dark at 4° C. Ringer solutions of drugs are made up fresh each day of use.

Compositions within the scope of this invention include all compositions wherein the compounds of the present invention are contained in an amount that is effective to achieve its intended purpose. While individual needs vary, determination of optimal ranges of effective amounts of each component is within the skill of the art. Typically, the compounds can be administered to mammals, e.g. humans, orally at a dose of 0.0025 to 50 mg/kg, or an equivalent amount of the pharmaceutically-acceptable salt thereof, per day of the body weight of the mammal being treated for epilepsy, neurodegenerative diseases, anesthetic, arrhythmia, manic depression and/or chronic pain. For intramuscular injection, the dose is generally about one-half of the oral dose.

In the method of treatment or prevention of neuronal loss in global and focal ischemia, brain and spinal cord trauma, hypoxia, hypoglycemia, status epilepsy and surgery, the compound can be administrated by intravenous injection at a dose of about 0.025 to about 10 mg/kg.

The unit oral dose can comprise from about 0.01 to about 50 mg, preferably about 0.1 to about 10 mg of the compound. The unit dose can be administered one or more times daily as one or more tablets each containing from about 0.1 to about 10, conveniently about 0.25 to about 50 mg of the compound or its solvate(s).

The following non-limiting examples are illustrative of the aspects of the present invention. Other suitable modifications and adaptations of the variety of conditions and parameters normally encountered in clinical therapy and which are obvious to those skilled in the art are within the spirit and scope of the invention.

### EXAMPLE 1

### N-(2-piperidin-1-ylethyl)-N-[3-(3-trifluoromethylphenoxy)benzyl]acetamide (5a)

N-(2-piperidin-1-ylethyl)-N-[3-(3-trifluoromethylphenoxy)benzyl]-amine (3): To a solution of 3-[3-(trifluoromethyl)-phenoxy]benzaldehyde (1, 4.0 g, 15.0 mmol) in THF (100 mL) was added 1-(2-aminoethyl)-piperidine (2, 2.3 g 18.0 mmol) and titanium (IV) isopropoxide (8.5 g, 30.0 mmol). After stirring 6 hours at ambient temperature, a solution of sodium borohydride (30.0 mmol) in ethanol (100 mL) was added, and the reaction was stirred for 24 hours. The reaction was then quenched with aqueous ammonia, and the resulting inorganic precipitate was filtered and washed with dichloromethane. The phases were separated, and the aqueous phase was extracted twice with dichloromethane. The combined organic phases were dried over sodium sulfate. The solution was filtered and then concentrated to give compound 3 as a pale-yellow oil. Purification of compound 3 was then carried out by silica gel chromatography.

To a solution of compound 3 (180 mg, 0.4 mmol) in methylene chloride (5 mL), acetyl chloride (0.6 mmol) and pyridine (0.8 mmol), were added. The reaction mixture was stirred for 2 hours and resulted in the formation of compound 5a, which was purified by silica gel chromatography.

N-(2-piperidin-1-ylethyl)-N-[3-(3-trifluoromethylphenoxy)benzyl]-cyclopentane carboxamide (5b) and N-(2-piperidin-1-ylethyl)-N-[3-(3-trifluoromethylphenoxy)benzyl]-4-fluorobenzamide (5c) were prepared analogously, replacing acetyl chloride with cyclopentanecarboxylic acid chloride and 4-fluorobenzoyl chloride, respectively.

### EXAMPLE 2

### N′-cyclohexyl-N-(2-piperidin-1-ylethyl)-N-[3-(3-trifluoromethylphenoxy)benzyl]urea (7a)

To a solution of compound 3 (180 mg, 0.4 mmol) in methylene chloride (5 mL), cyclohexylisocyanate (0.6 mmol), was added. The reaction mixture was stirred for 2 hours and resulted in the formation of compound 7a, which was purified by silica gel chromatography.

N′-(2-fluorophenyl)-N-(2-piperidin-1-ylethyl)-N-[3-(3-trifluoromethyl-phenoxy)benzyl]urea (7b) was prepared analogously, replacing cyclohexylisocyanate with 2-fluorophenylisocyanate.

### EXAMPLE 3

### N′-[3-(morpholin-4-yl)propyl]-N-(2-piperidin-1-ylethyl)-N-[3-(3-trifluoromethylphenoxy)benzyl]thiourea (7c)

To a solution of compound 3 (180 mg, 0.4 mmol) in methylene chloride (5 mL), 3-(morpholin-4-yl)propylisothiocyanate (0.6 mmol), was added. The reaction mixture was stirred for 2 hours and resulted in the formation of compound 7c, which was purified by silica gel chromatography.

### EXAMPLE 4

### Physical Data and Biological Activity of Compounds of the Present Invention

Physical data for compounds of the present invention are presented in Table 1. Using the above-described assay, the Ki values for sodium channel inhibition of compounds exemplified in Table 1 below, were determined to range from 70 to 4660 nM.

TABLE 1
PHYSICAL PROPERTIES FOR
AMINOALKYL-SUBSTITUTED ARYL COMPOUNDS

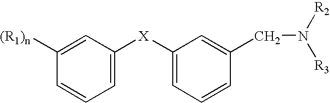

| Cmpd^{†} | R₂ | R₃ | NMR Data |
|---|---|---|---|
| 3 | 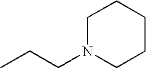 | —H | ¹H NMR(400 MHz, CDCl₃): δ1.39-1.40 (m, 2H), 1.49-1.54(m, 4H), 1.88(bs, 1H), 2.32(bs, 4H), 2.42(t, 2H), 2.67(t, 2H), 3.79(s, 2H), 6.89(d, 1H), 7.01(d, 1H), 7.11(t, 2H), 7.22(s, 1H), 7.30(t, 2H), 7.40(t, 1H). |
| 5a | 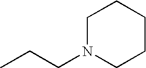 | 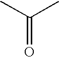 | ¹H NMR(400 MHz, CDCl₃): δ1.38-1.40 (m, 2H), 1.49-1.54(m, 4H), 2.07 and 2.16(pair of s, due to rotamers, 3H), 2.33-2.41(m, 4H), 2.39 and 2.48(pair of t, due to rotamers, 2H), 3.32 and 3.47 (pair of t, due to rotamers, 2H), 4.58 and 4.61(pair of s, due to rotamers, 2H), 6.85-6.88(m, 2H), 6.95(dd, 1H), 7.12(t, 1H), 7.19(d, 1H), |
| #7.25-7.32(m, 2H), 7.41(dd, 1H). | | | |
| 5b | 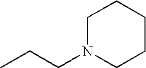 | 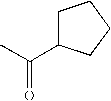 | ¹H NMR(400 MHz, CDCl₃): δ1.39(bm, 2H), 1.50-1.55(m, 6H), 1.71-1.79(m, 6H), 2.35-2.43(m, 4H), 2.41 and 2.45 (pair of t, due to rotamers, 2H), 2.80 and 2.92(pair of t, due to rotamers, 1H), 3.37 and 3.48(pair of t, due to rotamers, 2H), 4.61 and 4.64(pair of s, due to rotamers, 2H), 6.82(s, 1H), 6.89 and 6.97(pair of dd, due |
| #to rotamers, 2H), 7.10(bm, 1H), 7.26(d, 1H), 7.27-7.42 (m, 3H). | | | |
| 5c | 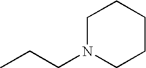 | 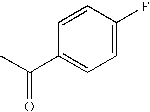 | ¹H NMR(400 MHz, CDCl₃): δ1.25-1.49 (m & bs, 6H), 2.15-2.59(m & bs, 6H), 3.30-3.55(m & bs, 2H), 4.61-4.77(m & bs, 4H), 6.83-7.25(m, 7H), 7.36-7.44 (m, 5H). |
| 7a | 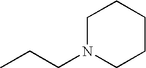 | 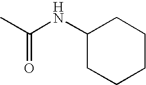 | ¹H NMR(400 MHz, CDCl₃): δ1.06-1.13 (m, 3H), 1.29-1.41(m, 4H), 1.51-1.57 (m, 5H), 1.68(d, 2H), 1.92(d, 2H), 2.27-2.34(m, 6H), 3.16(t, 2H), 3.56(m, 1H), 4.45(s, 2H), 6.86(d, 1H), 6.92(s, 1H), 7.04-7.10(m, 3H), 7.16(s, 1H), 7.25(m, 2H), 7.37(d, 1H). |
| 7b | 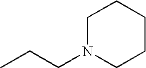 | 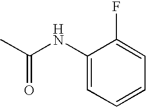 | ¹H NMR(400 MHz, CDCl₃): δ1.41-1.43 (m, 2H), 1.52-1.58(m, 4H), 2.45-2.47 (m, 6H), 3.38(t, 2H), 4.58(s, 2H), 6.86-7.08 (m, 5H), 7.15(d, 2H), 7.22(s, 1H), 7.31-7.35(m, 2H), 7.42(t, 1H), 7.81(t, 1H). |
| 7c | 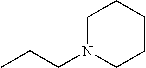 | 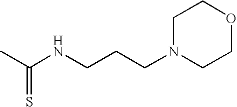 | ¹H NMR(400 MHz, CDCl₃): δ1.39-1.40 (m, 2H), 1.49-1.52(m, 4H), 1.82(t, 2H), 2.31-2.38(m, 6H), 3.36(t, 2H), 3.61-3.66 (m, 6H), 5.10(s, 2H), 6.85(d, 1H), 6.96(s, 1H), 7.09(d, 2H), 7.14(s, 1H), 7.26(t, 2H), 7.37(t, 1H). |

^{†}For all compounds in Table 1, X is —O—, n is 1 and R₁ is —CF₃.

### EXAMPLE 5

### Tablet Preparation

Tablets containing 25.0, 50.0, and 100.0 mg, respectively, of the compound of the invention (i.e., “active compound”) are prepared as illustrated in Table 2, below.

TABLE 2
TABLET FOR DOSES CONTAINING FROM
25-100 MG OF THE ACTIVE COMPOUND

| | Amount (mg) | | |
|---|---|---|---|
| Active compound | 25.0 | 50.0 | 100.0 |
| Microcrystalline cellulose | 37.25 | 100.0 | 200.0 |
| Modified food corn starch | 37.25 | 4.25 | 8.5 |
| Magnesium stearate | 0.50 | 0.75 | 1.5 |

All of the active compound, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of active ingredient per tablet. The specific amounts of each ingredient described in Table 2 are not intended to be limiting, but are rather exemplary. The amount of active ingredient can be any amount in the range of about 25 to about 100 mg. The amounts of the remaining ingredients can thus be adjusted accordingly, as deemed necessary by those of ordinary skill in the art.

### EXAMPLE 6

### Intravenous Solution Preparation

An intravenous dosage form of the compound of the invention (i.e., “active compound”) is prepared as shown in Table 3, below.

TABLE 3
INTRAVENOUS SOLUTION FORMULATION

| | | |
|---|---|---|
| Active compound | 0.5-10.0 | mg |
| Sodium citrate | 5-50 | mg |
| Citric acid | 1-15 | mg |
| Sodium chloride | 1-8 | mg |
| Water for injection (USP) | q.s. to 1 | mL |

Utilizing the above quantities, the active compound is dissolved at room temperature in a previously-prepared solution of sodium chloride, citric acid, and sodium citrate in Water for Injection (USP, see page 1636 of United States Pharmacopeia/National Formulary for 1995, published by United States Pharmacopeial Convention, Inc., Rockville, Md. (1994)).

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All documents (e.g., scientific publications, patents and patent publications) recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

1. A compound of Formula I: 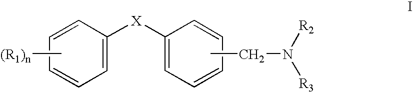
or a pharmaceutically-acceptable salt or solvate thereof, wherein:
R₁ is at each occurrence selected from the group consisting of halogen, optionally-substituted C₁₋₆ alkyl, amino, nitro and cyano;
n is an integer from 1 to 3;
X is —O—, —S—, —NH—, —NHCH₂—, —CH₂NH—, —CH₂—, —CH₂O—, —OCH₂—, —CH₂S— or —SCH₂—;
R₂ is 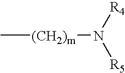
wherein:
m is an integer from 2 to 4;
R₄ and R₅ are independently selected from the group consisting of hydrogen and optionally-substituted C₁₋₆ alkyl; or R₄ and R₅ together with the nitrogen to which they are attached form a ring having 4 or 5 carbon atoms, which ring optionally contains 1 or 2 additional heteroatoms independently selected from —O—, —S—, and —NR₆—, wherein R₆ is hydrogen, C₁₋₆ alkyl C₁₋₆ haloalkyl or C₁₋₆ hydroxyalkyl; and
R₃ is hydrogen, optionally-substituted C₁₋₆ alkyl, 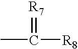
wherein:
R₇ is oxygen or sulfur; and
R₈ is selected from the group consisting of optionally-substituted C₁₋₆ alkyl, an optionally-substituted C₃₋₈ carbocyclic ring system and optionally-substituted C₆₋₁₀ aryl,
or R₃ is 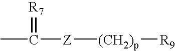
wherein:
R₇ is oxygen or sulfur;
Z is —O— or —NH—;
p is an integer from 0 to 4; and
R₉ is selected from the group consisting of optionally-substituted C₁₋₆ alkyl, an optionally-substituted C₃₋₈ carbocyclic ring system, optionally-substituted C₆₋₁₀ aryl, optionally-substituted heteroaryl and optionally-substituted heterocycle, wherein the heterocycle is saturated or partially unsaturated;
provided that,
when R₄ and R₅ are independently hydrogen or C₁₋₂ alkyl, or when R₄ and R₅ together with the nitrogen to which they are attached form pyrrolidinyl, then X is not —S—;
when X is —CH₂O— and R₃ is hydrogen or methyl, then at least one of R₄ or R₅ is not C₃₋₅ alkyl; and
when X is —O— and R₄ and R₅ together with the nitrogen to which they are attached form a ring having 4 or 5 carbon atoms, then R₈ is not a C₃ carbocyclic ring system, and R₉ is not C₃₋₅ alkyl, phenyl, dihalophenyl or (C₁₋₂ alkyl)phenyl.

2. The compound according to claim 1, wherein n is 1.

3. The compound according to claim 1, wherein R₁ is positioned meta relative to X.

4. The compound according to claim 1, wherein R₁ is C₁₋₆ haloalkyl.

5. The compound according to claim 1, wherein X include —O—.

6. The compound according to claim 1, wherein —CH₂—NR₂R₃ is positioned meta relative to X.

7. The compound according to claim 1, wherein m is 2.

8. The compound according to claim 1, wherein R₄ and R₅ together with the nitrogen to which they are attached form a piperidyl ring.

9. The compound according to claim 1, wherein R₃ is hydrogen or optionally-substituted C₁₋₆ alkyl.

10. The compound according to claim 9, wherein R₃ is hydrogen.

11. The compound according to claim 1, wherein R₃ is

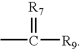

12. The compound according to claim 11, wherein R₇ is oxygen.

13. The compound according to claim 11, wherein R₈ is C₁₋₆ alkyl, C₅₋₆ cycloalkyl or optionally-substituted phenyl; wherein said optionally-substituted phenyl is optionally substituted once with halogen or C₁₋₄ alkyl.

14. The compound according to claim 11, wherein R₈ is acetyl, cyclopentanecarbonyl or p-fluorobenzoyl.

15. The compound according to claim 1, wherein R₃ is

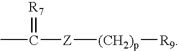

16. The compound according to claim 15, wherein R₇ is oxygen.

17. The compound according to claim 15, wherein Z is —NH—.

18. The compound according to claim 15, wherein p is zero, 1, 2 or 3.

19. The compound according to claim 15, wherein R₉ is C₅₋₆ cycloalkyl, optionally-substituted phenyl and 5- to 6-membered saturated or partially unsaturated heterocycle, wherein said optionally-substituted phenyl is optionally substituted once with halogen or C₁₋₄ alkyl.

20. The compound according to claim 15, wherein R₃ is cyclohexylamino-carbonyl, 2-fluorophenylaminocarbonyl or 3-(morpholin-4-yl)-propylamino-thiocarbonyl.

21. The compound according to claim 1, wherein:
R₃ is hydrogen or optionally-substituted C₁₋₆ alkyl;
R₄ and R₅ together with the nitrogen to which they are attached form a ring having 4 or 5 carbon atoms, which ring optionally contains 1 or 2 additional heteroatoms independently selected from the group consisting of —O—, —S— and —NR₆—, wherein R₆ is hydrogen, C₁₋₆ alkyl, C₁₋₆ haloalkyl or C₁₋₆ hydroxyalkyl; and
X is —O—, —CH₂—O— or —CH₂—S—.

22. The compound according to claim 1, wherein:
R₃ is hydrogen, C₁₋₆ alkyl, C₁₋₆ haloalkyl, C₁₋₆ hydroxyalkyl or C₁₋₆ alkyloxy(C₁₋₆)alkyl;
R₄ and R₅ together with the nitrogen to which they are attached form a ring having 4 or 5 carbon atoms, which ring optionally contains 1 or 2 additional heteroatoms selected from the group consisting of —O—, —S—, and —NR₆—, wherein R₆ is hydrogen or C₁₋₆ alkyl;
n is 1 or 2;
R₁ is halogen, C₁₋₆ alkyl or C₁₋₆ haloalkyl;
X is —O— or —CH₂—O—; and
m is 2 or 3.

23. The compound according to claim 1, wherein:
R₃ is hydrogen or C₁₋₆ alkyl;
R₄ and R₅ together with the nitrogen to which they are attached form a ring having 4 or 5 carbon atoms, which ring optionally contains 1 additional heteroatom independently selected from the group consisting of —O—, —S— and —NR₆—, wherein R₆ is hydrogen, C₁₋₆ alkyl, C₁₋₆ haloalkyl or C₁₋₆ hydroxyalkyl;
n is 1;
R₁ is C₁₋₆ haloalkyl;
X is —O—; and
m is 2.

24. The compound according to claim 1, wherein:
R₃ is hydrogen or optionally-substituted C₁₋₆ alkyl;
one of R₄ or R₅ is selected from the group consisting of hydrogen and optionally-substituted C₁₋₆ alkyl, and the other is selected from the group consisting of hydrogen, optionally-substituted C₁₋₂ alkyl and optionally-substituted C₆ alkyl; and
X is —O—, —CH₂—O— or —CH₂—S—.

25. The compound according to claim 1, wherein:
R₃ is hydrogen, C₁₋₆ alkyl, C₁₋₆ haloalkyl, C₁₋₆ hydroxyalkyl or C₁₋₆ alkyloxy(C₁₋₆)alkyl;
one of R₄ or R₅ is selected from the group consisting of hydrogen, C₁₋₆ alkyl and C₁₋₆ haloalkyl, and the other is selected from the group consisting of hydrogen, C₁₋₂ alkyl, C₆ alkyl, C₁₋₂ haloalkyl and C₆ haloalkyl;
n is 1 or 2;
R₁ is halogen, C₁₋₆ alkyl or C₁₋₆ haloalkyl;
X is —O— or —CH₂—O—; and
m is 2 or 3.

26. The compound according to claim 1, wherein:
R₃ is hydrogen or C₁₋₆ alkyl;
R₄ and R₅ are independently selected from the group consisting of hydrogen and C₁₋₆ alkyl;
n is 1;
R₁ is C₁₋₆ haloalkyl;
X is —O—; and
m is 2.

27. The compound according to claim 1, wherein R₃ is 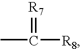
wherein:
R₇ is oxygen or sulfur;
R₈ is optionally-substituted C₁₋₆ alkyl, optionally-substituted C₅₋₈ cycloalkyl or optionally-substituted C₆₋₁₀ aryl;
R₄ and R₅ together with the nitrogen to which they are attached form a ring having 4 or 5 carbon atoms, which ring optionally contains 1 or 2 additional heteroatoms independently selected from the group consisting of —O—, —S— and —NR₆—, wherein R₆ is hydrogen, C₁₋₆ alkyl, C₁₋₆ haloalkyl or C₁₋₆ hydroxyalkyl; and
X is —O—, —CH₂—O— or —CH₂—S—.

28. The compound according to claim 1, wherein R₃ is 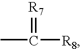
wherein:
R₇ is oxygen;
R₈ is C₁₋₆ alkyl, C₁₋₆ haloalkyl, C₁₋₆ hydroxyalkyl, optionally-substituted C₅₋₆ cycloalkyl or optionally-substituted phenyl;
R₄ and R₅ together with the nitrogen to which they are attached form a ring having 4 or 5 carbon atoms, which ring optionally contains 1 or 2 additional heteroatoms selected from the group consisting of —O—, —S—, and —NR₆—, wherein R₆ is hydrogen or C₁₋₆ alkyl;
n is 1 or 2;
R₁ is halogen, C₁₋₆ alkyl or C₁₋₆ haloalkyl;
X is —O— or —CH₂—O—; and
m is 2 or 3.

29. The compound according to claim 1, wherein R₃ is 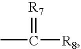
wherein:
R₇ is oxygen;
R₈ is C₁₋₆ alkyl, C₅₋₆ cycloalkyl or phenyl, wherein the phenyl is substituted with zero, 1 or 2 groups independently selected from the group consisting of halogen, C₁₋₆ alkyl, C₁₋₆ haloalkyl, —C₁₋₆ hydroxyalkyl, C₁₋₆ alkyloxy(C₁₋₆)alkyl, amino(C₁₋₆)alkyl, hydroxy, nitro and amino;
R₄ and R₅ together with the nitrogen to which they are attached form a ring having 4 or 5 carbon atoms, which ring optionally contains 1 additional heteroatom independently selected from the group consisting of —O—, —S— and —NR₆—, wherein R₆ is hydrogen, C₁₋₆ alkyl, C₁₋₆ haloalkyl or C₁₋₆ hydroxyalkyl;
n is 1;
R₁ is C₁₋₆ haloalkyl;
X is —O—; and
m is 2.

30. The compound according to claim 1, wherein R₃ is 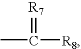
wherein:
R₇ is oxygen or sulfur;
R₈ is optionally-substituted C₁₋₆ alkyl, optionally-substituted C₃₋₈ cycloalkyl or optionally-substituted C₆₋₁₀ aryl;
R₄ and R₅ are independently selected from the group consisting of hydrogen and optionally-substituted C₁₋₆ alkyl; and
X is —O—, —CH₂—O— or —CH₂—S—.

31. The compound according to claim 1, wherein R₃ is 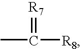
wherein:
R₇ is oxygen;
R₈ is C₁₋₆ alkyl, C₁₋₆ haloalkyl, C₁₋₆ hydroxyalkyl, optionally-substituted C₅₋₆ cycloalkyl or optionally-substituted phenyl;
R₄ and R₅ are independently selected from hydrogen, C₁₋₆ alkyl and C₁₋₆ haloalkyl;
n is 1 or 2;
R₁ is halogen, C₁₋₆ alkyl or C₁₋₆ haloalkyl;
X is —O— or —CH₂—O—; and
m is 2 or 3.

32. The compound according to claim 1, wherein R₃ is 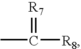
wherein:
R₇ is oxygen;
R₈ is C₁₋₆ alkyl, C₅₋₆ cycloalkyl or phenyl, wherein the phenyl is substituted with zero, 1 or 2 groups independently selected from the group consisting of halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino;
R₄ and R₅ are independently selected from the group consisting of hydrogen and C₁₋₆ alkyl;
n is 1;
R₁ is C₁₋₆ haloalkyl;
X is —O—; and
m is 2.

33. The compound according to claim 1, wherein R₃ is 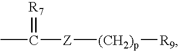
wherein:
R₉ is optionally-substituted C₁₋₂ alkyl, optionally-substituted C₆ alkyl, optionally-substituted C₃₋₈ cycloalkyl, substituted C₆₋₁₀ aryl, optionally-substituted heteroaryl or optionally-substituted saturated or partially unsaturated heterocycle;
R₄ and R₅ together with the nitrogen to which they are attached form a ring having 4 or 5 carbon atoms, which ring optionally contains 1 or 2 additional heteroatoms independently selected from the group consisting of —O—, —S— and —NR₆—, wherein R₆ is hydrogen, C₁₋₆ alkyl, C₁₋₆ haloalkyl or C₁₋₆ hydroxyalkyl; and
X is —O—, —CH₂—O— or —CH₂—S—.

34. The compound according to claim 1, wherein R₃ is 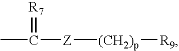
wherein:
R₇ is oxygen;
Z is —NH—;
p is zero, 1, 2 or 3;
R₉ is C₁₋₂ alkyl, C₆ alkyl, C₁₋₆ haloalkyl, C₁₋₆ hydroxyalkyl, optionally-substituted C₅₋₆ cycloalkyl, substituted phenyl or optionally-substituted 5- to 6-membered saturated or partially unsaturated heterocycle;
R₄ and R₅ together with the nitrogen to which they are attached form a ring having 4 or 5 carbon atoms, which ring optionally contains 1 or 2 additional heteroatoms selected from the group consisting of —O—, —S—, and —NR₆—, wherein R₆ is hydrogen or C₁₋₆ alkyl;
n is 1 or 2;
R₁ is halogen, C₁₋₆ alkyl or C₁₋₆ haloalkyl;
X is —O— or —CH₂—O—; and
m is 2 or 3.

35. The compound according to claim 1, wherein R₃ is 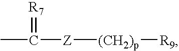
wherein:
R₇ is oxygen;
Z is —NH—;
p is zero, 1, 2 or 3;
R₉ is C₅₋₆ cycloalkyl, substituted phenyl or 5- to 6-membered saturated or partially unsaturated heterocycle, where the substituted phenyl is phenyl substituted with 1 or 2 groups independently selected from the group consisting of halogen, C₃₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino, and where the substituted phenyl is not dihalophenyl;
R₄ and R₅ together with the nitrogen to which they are attached form a ring having 4 or 5 carbon atoms, which ring optionally contains 1 additional heteroatom independently selected from —O—, —S— and —NR₆—, wherein R₆ is hydrogen, C₁₋₆ alkyl, C₁₋₆ haloalkyl or C₁₋₆ hydroxyalkyl; n is 1;
R₁ is C₁₋₆ haloalkyl;
X is —O—; and
m is 2.

36. The compound according to claim 1, wherein R₃ is 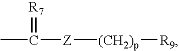
wherein:
R₉ is optionally-substituted C₁₋₆ alkyl, optionally-substituted C₃₋₈ cycloalkyl, optionally-substituted C₆₋₁₀ aryl, optionally-substituted heteroaryl or optionally-substituted saturated or partially unsaturated heterocycle;
R₄ and R₅ are independently selected from the group consisting of hydrogen and optionally-substituted C₁₋₆ alkyl; and
X is —O—, —CH₂—O— or —CH₂—S—.

37. The compound according to claim 1, wherein R₃ is 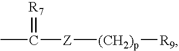
wherein:
R₇ is oxygen;
Z is —NH—;
p is zero, 1, 2 or 3;
R₉ is C₁₋₆ alkyl, C₁₋₆ haloalkyl, C₁₋₆ hydroxyalkyl, optionally-substituted C₅₋₆ cycloalkyl, optionally-substituted phenyl or optionally-substituted 5- to 6-membered saturated or partially unsaturated heterocycle;
R₄ and R₅ are independently selected from the group consisting of hydrogen, C₁₋₆ alkyl and C₁₋₆ haloalkyl;
n is 1 or 2;
R₁ is halogen, C₁₋₆ alkyl or C₁₋₆ haloalkyl;
X is —O— or —CH₂—O—; and
m is 2 or 3.

38. The compound according to claim 1, wherein R₃ is 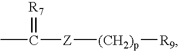
wherein:
R₇ is oxygen;
Z is —NH—,
p is zero, 1, 2 or 3;
R₉ is C₅₋₆ cycloalkyl, optionally-substituted phenyl or 5- to 6-membered saturated or partially unsaturated heterocycle, wherein the phenyl is substituted with zero, 1 or 2 groups independently selected from the group consisting of halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino;
R₄ and R₅ are independently selected from the group consisting of hydrogen and C₁₋₆ alkyl;
n is 1;
R₁ is C₁₋₆ haloalkyl;
X is —O—; and
m is 2.

39. A compound selected from the group consisting of
N-(2-piperidin-1-ylethyl)-N-[3-(3-trifluoromethylphenoxy)benzyl]amine;
N-(2-piperidin-1-ylethyl)-N-[3-(3-trifluoromethylphenoxy)benzyl]-acetamide;
N-(2-piperidin-1-ylethyl)-N-[3-(3-trifluoromethylphenoxy)benzyl]-cyclopentane carboxamide;
N-(2-piperidin-1-ylethyl)-N-[3-(3-trifluoromethylphenoxy)benzyl]-4-fluorobenzamide;
N′-cyclohexyl-N-(2-piperidin-1-ylethyl)-N-[3-(3-trifluoromethylphenoxy)-benzyl]urea;
N′-(2-fluorophenyl)-N-(2-piperidin-1-ylethyl)-N-[3-(3-trifluoromethyl-phenoxy)benzyl]urea; and
N′-[3-(morpholin-4-yl)propyl]-N-(2-piperidin-1-ylethyl)-N-[3-(3-trifluoromethylphenoxy)benzyl]thiourea;
and pharmaceutically-acceptable salts thereof.

40. A pharmaceutical composition comprising the compound according to claim 1, or a pharmaceutically-acceptable salt thereof, and a pharmaceutically-acceptable carrier or diluent.

41. A method for treating, preventing or ameliorating a disorder responsive to blockage of sodium ion channels in a mammal suffering therefrom, comprising administering to said mammal in need of such treatment an effective amount of a compound of Formula I 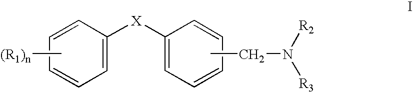
or a pharmaceutically-acceptable salt or solvate thereof, wherein:
R₁ is at each occurrence independently selected from the group consisting of hydrogen, halogen, optionally-substituted C₁₋₆ alkyl, amino, nitro and cyano;
n is an integer from 1 to 3;
X is —O—, —S—, —NH—, —NHCH₂—, —CH₂NH—, —CH₂—, —CH₂O—, —OCH₂—, —CH₂S— or —SCH₂—;
R₂ is 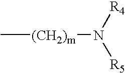
wherein:
m is an integer from 2 to 4;
R₄ and R₅ are independently selected from hydrogen and optionally-substituted C₁₋₆ alkyl; or R₄ and R₅ together with the nitrogen to which they are attached form a ring having 4 or 5 carbon atoms, which ring optionally contains 1 or 2 additional heteroatoms independently selected from —O—, —S—, and —NR₆—, wherein R₆ is hydrogen, C₁₋₆ alkyl, C₁₋₆ haloalkyl or C₁₋₆ hydroxyalkyl; and
R₃ is hydrogen, optionally-substituted C₁₋₆ alkyl, 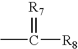
wherein:
R₇ is oxygen or sulfur; and
R₈ is selected from optionally-substituted C₁₋₆ alkyl, an optionally-substituted C₃₋₈ carbocyclic ring system and optionally-substituted C₆₋₁₀ aryl, or R₃ is 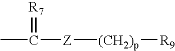
wherein:
R₇ is oxygen or sulfur;
Z is —O— or —NH—;
p is an integer from zero to 4; and
R₉ is selected from optionally-substituted C₁₋₆ alkyl, an optionally-substituted C₃₋₈ carbocyclic ring system, optionally-substituted C₆₋₁₀ aryl, optionally-substituted heteroaryl and optionally-substituted heterocycle, wherein the heterocycle is saturated or partially unsaturated.

42. The method according to claim 41, wherein:
R₃ is hydrogen or optionally-substituted C₁₋₆ alkyl;
R₄ and R₅ together with the nitrogen to which they are attached form a ring having 4 or 5 carbon atoms, which ring optionally contains 1 or 2 additional heteroatoms independently selected from the group consisting of —O—, —S— and —NR₆—, wherein R₆ is hydrogen, C₁₋₆ alkyl, C₁₋₆ haloalkyl or C₁₋₆ hydroxyalkyl; and
X is —O—, —S—, —CH₂—O— or —CH₂—S—.

43. The method according to claim 41, wherein:
R₃ is hydrogen, C₁₋₆ alkyl, C₁₋₆ haloalkyl, C₁₋₆ hydroxyalkyl or C₁₋₆ alkyloxy(C₁₋₆)alkyl;
R₄ and R₅ together with the nitrogen to which they are attached form a ring having 4 or 5 carbon atoms, which ring optionally contains 1 or 2 additional heteroatoms selected from the group consisting of —O—, —S—, and —NR₆—, wherein R₆ is hydrogen or C₁₋₆ alkyl;
n is 1 or 2;
R₁ is halogen, C₁₋₆ alkyl or C₁₋₆ haloalkyl;
X is —O— or —S—; and
m is 2 or 3.

44. The method according to claim 41, wherein:
R₃ is hydrogen or C₁₋₆ alkyl;
R₄ and R₅ together with the nitrogen to which they are attached form a ring having 4 or 5 carbon atoms, which ring optionally contains 1 additional heteroatom independently selected from the group consisting of —O—, —S— and —NR₆—, wherein R₆ is hydrogen, C₁₋₆ alkyl, C₁₋₆ haloalkyl or C₁₋₆ hydroxyalkyl;
n is 1;
R₁ is C₁₋₆ haloalkyl;
X is —O—; and
m is 2.

45. The method according to claim 41, wherein:
R₃ is hydrogen or optionally-substituted C₁₋₆ alkyl;
R₄ and R₅ are independently selected from the group consisting of hydrogen and optionally-substituted C₁₋₆ alkyl; and
X is —O—, —S—, —CH₂—O— or —CH₂—S—.

46. The method according to claim 41, wherein:
R₃ is hydrogen, C₁₋₆ alkyl, C₁₋₆ haloalkyl, C₁₋₆ hydroxyalkyl or C₁₋₆ alkyloxy(C₁₋₆)alkyl;
R₄ and R₅ are independently selected from the group consisting of hydrogen, C₁₋₆ alkyl and C₁₋₆ haloalkyl;
n is 1 or 2;
R₁ is halogen, C₁₋₆ alkyl or C₁₋₆ haloalkyl;
X is —O— or —S—; and
m is 2 or 3.

47. The method according to claim 41, wherein:
R₃ is hydrogen or C₁₋₆ alkyl;
R₄ and R₅ are independently selected from the group consisting of hydrogen and C₁₋₆ alkyl;
n is 1;
R₁ is C₁₋₆ haloalkyl;
X is —O—; and
m is 2.

48. The method according to claim 41, wherein R₃ is 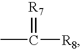
wherein:
R₇ is oxygen or sulfur;
R₈ is optionally-substituted C₁₋₆ alkyl, optionally-substituted C₃₋₈ cycloalkyl or optionally-substituted C₆₋₁₀ aryl;
R₄ and R₅ together with the nitrogen to which they are attached form a ring having 4 or 5 carbon atoms, which ring optionally contains 1 or 2 additional heteroatoms independently selected from the group consisting of the group consisting of —O—, —S— and —NR₆—, wherein R₆ is hydrogen, C₁₋₆ alkyl, C₁₋₆ haloalkyl or C₁₋₆ hydroxyalkyl; and
X is —O—, —S—, —CH₂—O— or —CH₂—S—.

49. The method according to claim 41, wherein R₃ is 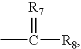
wherein:
R₇ is oxygen;
R₈ is C₁₋₆ alkyl, C₁₋₆ haloalkyl, C₁₋₆ hydroxyalkyl, optionally-substituted C₅₋₆ cycloalkyl or optionally-substituted phenyl;
R₄ and R₅ together with the nitrogen to which they are attached form a ring having 4 or 5 carbon atoms, which ring optionally contains 1 or 2 additional heteroatoms selected from the group consisting of —O—, —S—, and —NR₆—, wherein R₆ is hydrogen or C₁₋₆ alkyl;
n is 1 or 2;
R₁ is halogen, C₁₋₆ alkyl or C₁₋₆ haloalkyl;
X is —O— or —S—; and
m is 2, or 3.

50. The method according to claim 41, wherein R₃ is 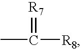
wherein:
R₇ is oxygen;
R₈ is C₁₋₆ alkyl, C₅₋₆ cycloalkyl or phenyl, wherein the phenyl is substituted with zero, 1 or 2, groups independently selected from the group consisting of halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino;
R₄ and R₅ together with the nitrogen to which they are attached form a ring having 4 or 5 carbon atoms, which ring optionally contains 1 additional heteroatom independently selected from the group consisting of —O—, —S— and —NR₆—, wherein R₆ is hydrogen, C₁₋₆ alkyl, C₁₋₆ haloalkyl or C₁₋₆ hydroxyalkyl;
n is 1;
R₁ is C₁₋₆ haloalkyl;
X is —O—; and
m is 2.

51. The method according to claim 41, wherein R₃ is 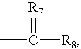
wherein:
R₇ is oxygen or sulfur;
R₈ is optionally-substituted C₁₋₆ alkyl, optionally-substituted C₃₋₈ cycloalkyl or optionally-substituted C₆₋₁₀ aryl;
R₄ and R₅ are independently selected from the group consisting of hydrogen and optionally-substituted C₁₋₆ alkyl; and
X is —O—, —S—, —CH₂—O— or —CH₂—S—.

52. The method according to claim 41, wherein R₃ is 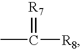
wherein:
R₇ is oxygen;
R₈ is C₁₋₆ alkyl, C₁₋₆ haloalkyl, C₁₋₆ hydroxyalkyl, optionally-substituted C₅₋₆ cycloalkyl or optionally-substituted phenyl;
R₄ and R₅ are independently selected from the group consisting of hydrogen, C₁₋₆ alkyl and C₁₋₆ haloalkyl;
n is 1 or 2;
R₁ is halogen, C₁₋₆ alkyl or C₁₋₆ haloalkyl;
X is —O— or —S—; and
m is 2 or 3.

53. The method according to claim 41, wherein R₃ is 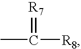
wherein:
R₇ is oxygen;
R₈ is C₁₋₆ alkyl, C₅₋₆ cycloalkyl or phenyl, wherein the phenyl is substituted with zero, 1 or 2 groups independently selected from the group consisting of halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino;
R₄ and R₅ are independently selected from the group consisting of hydrogen and C₁₋₆ alkyl;
n is 1;
R₁ is C₁₋₆ haloalkyl;
X is —O—; and
m is 2.

54. The method according to claim 41, wherein R₃ is 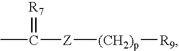
wherein:
R₉ is optionally-substituted C₁₋₆ alkyl, optionally-substituted C₃₋₈ cycloalkyl, optionally-substituted C₆₋₁₀ aryl, optionally-substituted heteroaryl or optionally-substituted saturated or partially unsaturated heterocycle;
R₄ and R₅ together with the nitrogen to which they are attached form a ring having 4 or 5 carbon atoms, which ring optionally contains 1 or 2 additional heteroatoms independently selected from the group consisting of —O—, —S— and —NR₆—, wherein R₆ is hydrogen, C₁₋₆ alkyl, C₁₋₆ haloalkyl or C₁₋₆ hydroxyalkyl; and
X is —O—, —S—, —CH₂—O— or —CH₂—S—.

55. The method according to claim 41, wherein R₃ is 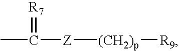
wherein:
R₇ is oxygen;
Z is —NH—;
p is zero, 1, 2 or 3;
R₉ is C₁₋₆ alkyl, C₁₋₆ haloalkyl, C₁₋₆ hydroxyalkyl, optionally-substituted C₅₋₆ cycloalkyl, optionally-substituted phenyl or optionally-substituted 5- to 6-membered saturated or partially unsaturated heterocycle;
R₄ and R₅ together with the nitrogen to which they are attached form a ring having 4 or 5 carbon atoms, which ring optionally contains 1 or 2 additional heteroatoms selected from the group consisting of —O—, —S—, and —NR₆—, wherein R₆ is hydrogen or C₁₋₆ alkyl;
n is 1 or 2;
R₁ is halogen, C₁₋₆ alkyl or C₁₋₆ haloalkyl;
X is —O— or —S—; and
m is 2 or 3.

56. The method according to claim 41, wherein R₃ is 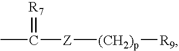
wherein:
R₇ is oxygen;
Z is —NH—;
p is zero, 1, 2 or 3;
R₉ is C₅₋₆ cycloalkyl, optionally-substituted phenyl or 5- to 6-membered saturated or partially unsaturated heterocycle, wherein the phenyl is substituted with zero, 1 or 2 groups independently selected from the group consisting of halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino;
R₄ and R₅ together with the nitrogen to which they are attached form a ring having 4 or 5 carbon atoms, which ring optionally contains 1 additional heteroatom independently selected from the group consisting of —O—, —S— and —NR₆—, wherein R₆ is hydrogen, C₁₋₆ alkyl, C₁₋₆ haloalkyl or C₁₋₆ hydroxyalkyl;
n is 1;
R₁ is C₁₋₆ haloalkyl;
X is —O—; and
m is 2.

57. The method according to claim 41, wherein R₃ is 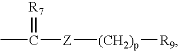
wherein:
R₉ is optionally-substituted C₁₋₆ alkyl, optionally-substituted C₃₋₈ cycloalkyl, optionally-substituted C₆₋₁₀ aryl, optionally-substituted heteroaryl or optionally-substituted saturated or partially unsaturated heterocycle;
R₄ and R₅ are independently selected from the group consisting of hydrogen and optionally-substituted C₁₋₆ alkyl; and
X is —O—, —S—, —CH₂—O— or —CH₂—S—.

58. The method according to claim 41, wherein R₃ is 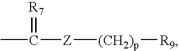
wherein:
R₇ is oxygen;
Z is —NH—;
p is zero, 1, 2 or 3;
R₉ is C₁₋₆ alkyl, C₁₋₆ haloalkyl, C₁₋₆ hydroxyalkyl, optionally-substituted C₅₋₆ cycloalkyl, optionally-substituted phenyl or optionally-substituted 5- to 6-membered saturated or partially unsaturated heterocycle;
R₄ and R₅ are independently selected from the group consisting of hydrogen, C₁₋₆ alkyl and C₁₋₆ haloalkyl;
n is 1 or 2;
R₁ is halogen, C₁₋₆ alkyl or C₁₋₆ haloalkyl;
X is —O— or —S—; and
m is 2 or 3.

59. The method according to claim 41, wherein R₃ is 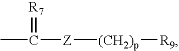
wherein:
R₇ is oxygen;
Z is —NH—;
p is zero, 1, 2 or 3;
R₉ is C₅₋₆ cycloalkyl, optionally-substituted phenyl or 5- to 6-membered saturated or partially unsaturated heterocycle, wherein the phenyl is substituted with zero, 1 or 2, preferably zero or one, groups independently selected from the group consisting of halogen, C₁₋₆ alkyl, C₁₋₆ haloalkyl, C₁₋₆ hydroxyalkyl, C₁₋₆ alkyloxy(C₁₋₆)alkyl, amino(C₁₋₆)alkyl, hydroxy, nitro and amino;
R₄ and R₅ are independently selected from the group consisting of hydrogen and C₁₋₆ alkyl;
n is 1;
R₁ is C₁₋₆ haloalkyl;
X is —O—; and
m is 2.

60. The method according to claim 41, wherein said disorder is selected from the group consisting of neuronal damage, acute or chronic pain, neuropathic pain, surgical pain, convulsions, a neurodegenerative condition, manic depression and diabetic neuropathy.

61. The method according to claim 41, wherein said disorder is acute or chronic pain.

62. The method according to claim 41, wherein said disorder is neuropathic pain.

63. The method according to claim 41, wherein said disorder is surgical pain.

64. The method according to claim 41, wherein said disorder is neuronal damage caused by focal or global ischemia.

65. The method according to claim 41, wherein said disorder is a neuro-degenerative condition.

66. The method according to claim 65, wherein said neurodegenerative condition is amyotrophic lateral sclerosis (ALS).

67. The method according to claim 41, wherein said compound functions as an antitinnitus agent, an anticonvulsant, an antiarrhythmic, a local anesthetic or an antimaniac depressant.

68. The method according to claim 41, wherein said mammal is a human, dog or cat.

69. The method according to claim 41, wherein said mammal is a human.

70. A method of making the compound according to claim 1, wherein said method comprises:
(a) reacting in a first step an aryl aldehyde with a primary or secondary amine;
(b) optionally, in a second step, reacting the product of the first step (i) with an acid chloride compound of Formula II: 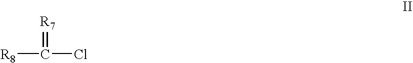
wherein R₇ is oxygen or sulfur, and R₉ is selected from the group consisting of optionally-substituted C₁₋₆ alkyl, an optionally-substituted C₃₋₈ carbocyclic ring system, or an optionally-substituted C₆₋₁₀ aryl;
(ii) with an isocyanate of Formula III: 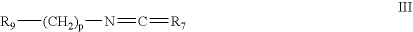
wherein p is an integer from zero to 4, R₇ is oxygen or sulfur, and R₉ is selected from the group consisting of optionally-substituted C₁₋₆ alkyl, an optionally-substituted C₃₋₈ carbocyclic ring system, optionally-substituted C₆₋₁₀ aryl, optionally-substituted heteroaryl and saturated or partially unsaturated heterocycle;
or (iii) with either triphosgene and triethylamine or thiophosgene and triethylamine, followed by R₉—(CH₂)ₚ—OH, wherein p is an integer from zero to 4, and R₉ is selected from the group consisting of optionally-substituted C₁₋₆ alkyl, an optionally-substituted C₃₋₈ carbocyclic ring system, optionally-substituted C₆₋₁₀ aryl, optionally-substituted heteroaryl and saturated or partially unsaturated heterocycle; and
(c) recovering the product obtained from either of the first or second steps.
